(19) United States
(12) Patent Application Publication
(10) Pub. No.: US 2025/0176878 A1
(43) Pub. Date: Jun. 5, 2025

(54) ANALYTE SENSOR MONITORING

(71) Applicant: Dexcom, Inc., San Diego, CA (US)

(72) Inventors: Yuxi Zhang, Santa Clara, CA (US); Liang Wang, San Diego, CA (US); Ghazaleh R Esmaili, San Diego, CA (US)

(21) Appl. No.: 18/968,829

(22) Filed: Dec. 4, 2024

Related U.S. Application Data
(60) Provisional application No. 63/605,935, filed on Dec. 4, 2023.

(51) Int. Cl. A61B 5/1495; A61B 5/145; G16H 10/60; G16H 40/67

(52) U.S. Cl. CPC A61B 5/1495; A61B 5/14532; G16H 10/60; G16H 40/67

(57) ABSTRACT

Various examples are directed to continuous analyte sensor systems and methods. A continuous analyte sensor system may execute a computerized model based on the sensor data. This may include providing a first portion of the sensor data from a first portion of the time period to the first LSTM layer, providing a second portion of the sensor data from a second portion of the time period to the second LSTM layer, the second portion of the time period being shorter than the first portion of the time period, and generating a predicted sensor state based on an output of the first LSTM layer and an output of the second LSTM layer.

### CLAIM FOR PRIORITY

This application claims priority to U.S. Provisional Application Ser. No. 63/605,935, filed Dec. 4, 2023, which is hereby incorporated by reference herein in its entirety.

### TECHNICAL FIELD

The present development relates generally to medical devices such as analyte sensors, and more particularly, but not by way of limitation, to systems, devices, and methods for operating an analyte sensor.

### BACKGROUND

Diabetes is a metabolic condition relating to the production or use of insulin by the body. Insulin is a hormone that allows the body to use glucose for energy, or store glucose as fat.

When a person eats a meal that contains carbohydrates, the food is processed by the digestive system, which produces glucose in the person's blood. Blood glucose can be used for energy or stored as fat. The body normally maintains blood glucose levels in a range that provides sufficient energy to support bodily functions and avoids problems that can arise when glucose levels are too high or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), and kidney damage. Hyperglycemia can also lead to acute problems, such as diabetic ketoacidosis-a state in which the body becomes excessively acidic due to the presence of blood glucose and ketones, which are produced when the body cannot use glucose. The state of having lower than normal blood glucose levels is called “hypoglycemia.” Severe hypoglycemia can lead to acute crises that can result in seizures or death.

A diabetes patient can receive insulin to manage blood glucose levels. Insulin can be received, for example, through a manual injection with a needle. Wearable insulin pumps are also available. Diet and exercise also affect blood glucose levels. A glucose sensor can provide an estimated glucose concentration level, which can be used as guidance by a patient or caregiver.

Diabetes conditions are sometimes referred to as “Type 1” and “Type 2.” A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Blood sugar concentration levels may be monitored with an analyte sensor, such as a continuous glucose monitor. A continuous glucose monitor is used by a host (e.g., patient) to provide information, such as an estimated blood glucose value or a trend of estimated blood glucose levels.

### SUMMARY

This present application discloses, among other things, systems, devices, and methods related to analyte sensor, including, for example, deploy testing in analyte sensors.

Example 1 is a continuous analyte sensor system comprising: a continuous analyte sensor; and sensor electronics in communication with the continuous analyte sensor, the sensor electronics being programmed to perform operations comprising: accessing sensor data generated by the continuous analyte sensor over a time period; executing a computerized model based on the sensor data, the computerized model comprising: a first Long Short-Term Memory (LSTM) layer and a second LSTM layer, the executing of the computerized model comprising: providing a first portion of the sensor data to the first LSTM layer, the first portion of the sensor data being from a first portion of the time period; providing a second portion of the sensor data to the second LSTM layer, the second portion of the sensor data being from a second portion of the time period, the second portion of the time period being shorter than the first portion of the time period, and the first portion of the time period beginning before the second portion of the time period; and generating a predicted sensor state based on an output of the first LSTM layer and an output of the second LSTM layer; and responsive to the predicted sensor state indicating an error condition, modifying a user interface output to suspend display of an output of the continuous analyte sensor.

In Example 2, the subject matter of Example 1 optionally includes hours.

In Example 3, the subject matter of any one or more of Examples 1-2 optionally includes minutes.

In Example 4, the subject matter of any one or more of Examples 1-3 optionally includes the period of time being about 30 seconds.

In Example 5, the subject matter of any one or more of Examples 1-4 optionally includes the output of the second LSTM layer being based on at least a portion of output of the first LSTM layer.

In Example 6, the subject matter of Example 5 optionally includes the computerized model further comprising a third LSTM layer, the executing of the computerized model further comprising providing a third portion of the sensor data to the third LSTM layer, the third portion of the sensor data being from a third portion of the time period, the third portion of the time period being shorter than the second portion of the time period, and the second portion of the time period beginning more recently than the third portion of the time period.

In Example 7, the subject matter of Example 6 optionally includes the predicted sensor state also being based on an output of the third LSTM layer.

In Example 8, the subject matter of Example 7 optionally includes the output of the third LSTM layer being based on at least a portion of the output of the first LSTM layer and at least a portion of the output of the second LSTM layer.

In Example 9, the subject matter of any one or more of Examples 7-8 optionally includes the executing of the computerized model further comprising executing a first fully connected layer, the first fully connected layer generating a first fully connected layer output based on the output of the first LSTM layer, the predicted sensor state also being based on the output of the first fully connected layer.

In Example 10, the subject matter of any one or more of Examples 1-9 optionally includes the operations further comprising, responsive to the predicted sensor state indicating an error condition, terminating a sensor session of the continuous analyte sensor.

In Example 11, the subject matter of any one or more of Examples 1-10 optionally includes the operations further comprising, responsive to the predicted sensor state indicating an error condition, ceasing to provide a bias to the continuous analyte sensor.

Example 12 is a method of operating a continuous analyte sensor, the method comprising: accessing sensor data generated by the continuous analyte sensor over a time period; executing a computerized model based on the sensor data, the computerized model comprising: a first Long Short-Term Memory (LSTM) layer and a second LSTM layer, the executing of the computerized model comprising: providing a first portion of the sensor data to the first LSTM layer, the first portion of the sensor data being from a first portion of the time period; providing a second portion of the sensor data to the second LSTM layer, the second portion of the sensor data being from a second portion of the time period, the second portion of the time period being shorter than the first portion of the time period, and the first portion of the time period beginning before the second portion of the time period; and generating a predicted sensor state based on an output of the first LSTM layer and an output of the second LSTM layer; and responsive to the predicted sensor state indicating an error condition, modifying a user interface output to suspend display of an output of the continuous analyte sensor.

In Example 13, the subject matter of Example 12 optionally includes hours.

In Example 14, the subject matter of any one or more of Examples 12-13 optionally includes minutes.

In Example 15, the subject matter of any one or more of Examples 12-14 optionally includes the period of time being about 30 seconds.

In Example 16, the subject matter of any one or more of Examples 12-15 optionally includes the output of the second LSTM layer being based on at least a portion of output of the first LSTM layer.

In Example 17, the subject matter of Example 16 optionally includes the computerized model further comprising a third LSTM layer, the executing of the computerized model further comprising providing a third portion of the sensor data to the third LSTM layer, the third portion of the sensor data being from a third portion of the time period, the third portion of the time period being shorter than the second portion of the time period, and the second portion of the time period beginning more recently than the third portion of the time period.

In Example 18, the subject matter of Example 17 optionally includes the predicted sensor state also being based on an output of the third LSTM layer.

In Example 19, the subject matter of Example 18 optionally includes the output of the third LSTM layer being based on at least a portion of the output of the first LSTM layer and at least a portion of the output of the second LSTM layer.

In Example 20, the subject matter of any one or more of Examples 18-19 optionally includes the executing of the computerized model further comprising executing a first fully connected layer, the first fully connected layer generating a first fully connected layer output based on the output of the first LSTM layer, the predicted sensor state also being based on the output of the first fully connected layer.

In Example 21, the subject matter of any one or more of Examples 12-20 optionally includes responsive to the predicted sensor state indicating an error condition, terminating a sensor session of the continuous analyte sensor.

In Example 22, the subject matter of any one or more of Examples 12-21 optionally includes responsive to the predicted sensor state indicating an error condition, ceasing to provide a bias to the continuous analyte sensor.

Example 23 is a non-transitory machine-readable medium comprising instructions thereon that, when executed by at least one processor, cause the at least one processor to perform operations comprising: accessing sensor data generated by A continuous analyte sensor over a time period; executing a computerized model based on the sensor data, the computerized model comprising: a first Long Short-Term Memory (LSTM) layer and a second LSTM layer, the executing of the computerized model comprising: providing a first portion of the sensor data to the first LSTM layer, the first portion of the sensor data being from a first portion of the time period; providing a second portion of the sensor data to the second LSTM layer, the second portion of the sensor data being from a second portion of the time period, the second portion of the time period being shorter than the first portion of the time period, and the first portion of the time period beginning before the second portion of the time period; and generating a predicted sensor state based on an output of the first LSTM layer and an output of the second LSTM layer; and responsive to the predicted sensor state indicating an error condition, modifying a user interface output to suspend display of an output of the continuous analyte sensor.

In Example 24, the subject matter of Example 23 optionally includes hours.

In Example 25, the subject matter of any one or more of Examples 23-24 optionally includes minutes.

In Example 26, the subject matter of any one or more of Examples 23-25 optionally includes the period of time being about 30 seconds.

In Example 27, the subject matter of any one or more of Examples 23-26 optionally includes the output of the second LSTM layer being based on at least a portion of output of the first LSTM layer.

In Example 28, the subject matter of any one or more of Examples 26-27 optionally includes the computerized model further comprising a third LSTM layer, the executing of the computerized model further comprising providing a third portion of the sensor data to the third LSTM layer, the third portion of the sensor data being from a third portion of the time period, the third portion of the time period being shorter than the second portion of the time period, and the second portion of the time period beginning more recently than the third portion of the time period.

In Example 29, the subject matter of Example 28 optionally includes the predicted sensor state also being based on an output of the third LSTM layer.

In Example 30, the subject matter of any one or more of Examples 28-29 optionally includes the output of the third LSTM layer being based on at least a portion of the output of the first LSTM layer and at least a portion of the output of the second LSTM layer.

In Example 31, the subject matter of any one or more of Examples 28-30 optionally includes the executing of the computerized model further comprising executing a first fully connected layer, the first fully connected layer generating a first fully connected layer output based on the output of the first LSTM layer, the predicted sensor state also being based on the output of the first fully connected layer.

In Example 32, the subject matter of any one or more of Examples 23-31 optionally includes the operations further comprising, responsive to the predicted sensor state indicating an error condition, terminating a sensor session of the continuous analyte sensor.

In Example 33, the subject matter of any one or more of Examples 23-32 optionally includes the operations further comprising, responsive to the predicted sensor state indicating an error condition, ceasing to provide a bias to the continuous analyte sensor.

Example 34 is a continuous analyte sensor system comprising: a continuous analyte sensor; and sensor electronics in communication with the continuous analyte sensor, the sensor electronics being programmed to perform operations comprising: accessing sensor data generated by the continuous analyte sensor over a time period; executing a computerized model based on the sensor data, the computerized model comprising a convolutional neural network (CNN), the CNN comprising a plurality of layers; generating a predicted sensor state based on an output of the computerized model; and responsive to the predicted sensor state indicating an error condition, modifying a user interface output to suspend display of an output of the continuous analyte sensor.

In Example 35, the subject matter of Example 34 optionally includes a first layer of the plurality of layers receiving an output of a first prior layer and an input to a second prior layer.

In Example 36, the subject matter of any one or more of Examples 34-35 optionally includes a first layer of the plurality of layers receiving an output of a first prior layer and an input to the first prior layer.

In Example 37, the subject matter of any one or more of Examples 34-36 optionally includes the operations further comprising training the CNN, the training of the CNN comprising: selecting a first set of dropout cells from at least one of the plurality of layers; deactivating the first set of dropout cells; executing the CNN using first training data, the CNN being configured with the first set of dropout cells deactivated during the executing; and modifying at least one layer of the CNN based on the executing of the CNN using the first training data.

In Example 38, the subject matter of Example 37 optionally includes the training of the CNN further comprising: re-activating the first set of dropout cells; selecting a second set of dropout cells from at least one of the plurality of layers; executing the CNN using second training data, the CNN being configured with the second set of dropout cells deactivated during the executing; and modifying at least one layer of the CNN based on the executing of the CNN using the second training data.

In Example 39, the subject matter of any one or more of Examples 37-38 optionally includes the deactivating of the first set of dropout cells comprising, for a first cell of the first set of dropout cells: modifying the CNN to remove an input to the first cell; and modifying the CNN to remove an output from the first cell.

In Example 40, the subject matter of any one or more of Examples 37-39 optionally includes the selecting of the first set of dropout cells comprising randomly or pseudo-randomly selecting a first cell from one of the plurality of layers.

In Example 41, the subject matter of any one or more of Examples 34-40 optionally includes the operations further comprising training the CNN, the training of the CNN comprising: executing the CNN using first training data; determining a model loss metric based on the executing of the CNN using the first training data; determining a model complexity metric based on the executing of the CNN using the first training data; and modifying at least one layer of the CNN, the modifying being based on the model loss metric and the model complexity metric.

In Example 42, the subject matter of any one or more of Examples 34-41 optionally includes the operations further comprising, responsive to the predicted sensor state indicating an error condition, terminating a sensor session of the continuous analyte sensor.

In Example 43, the subject matter of any one or more of Examples 34-42 optionally includes the operations further comprising, responsive to the predicted sensor state indicating an error condition, ceasing to provide a bias to the continuous analyte sensor.

Example 44 is a method of operating a continuous analyte sensor, the method comprising: accessing sensor data generated by the continuous analyte sensor over a time period; executing a computerized model based on the sensor data, the computerized model comprising a convolutional neural network (CNN), the CNN comprising a plurality of layers; generating a predicted sensor state based on an output of the computerized model; and responsive to the predicted sensor state indicating an error condition, modifying a user interface output to suspend display of an output of the continuous analyte sensor.

In Example 45, the subject matter of Example 44 optionally includes a first layer of the plurality of layers receiving an output of a first prior layer and an input to a second prior layer.

In Example 46, the subject matter of any one or more of Examples 44-45 optionally includes the CNN comprising the plurality of layers, a first layer of the plurality of layers receiving an output of a first prior layer and an input to the first prior layer.

In Example 47, the subject matter of any one or more of Examples 44-46 optionally includes training the CNN, the training of the CNN comprising: selecting a first set of dropout cells from at least one of the plurality of layers; deactivating the first set of dropout cells; executing the CNN using first training data, the CNN being configured with the first set of dropout cells deactivated during the executing; and modifying at least one layer of the CNN based on the executing of the CNN using the first training data.

In Example 48, the subject matter of Example 47 optionally includes the training of the CNN further comprising: re-activating the first set of dropout cells; selecting a second set of dropout cells from at least one of the plurality of layers; executing the CNN using second training data, the CNN being configured with the second set of dropout cells deactivated during the executing; and modifying at least one layer of the CNN based on the executing of the CNN using the second training data.

In Example 49, the subject matter of any one or more of Examples 47-48 optionally includes the deactivating of the first set of dropout cells comprising, for a first cell of the first set of dropout cells: modifying the CNN to remove an input to the first cell; and modifying the CNN to remove an output from the first cell.

In Example 50, the subject matter of any one or more of Examples 47-49 optionally includes the selecting of the first set of dropout cells comprising randomly or pseudo-randomly selecting a first cell from one of the plurality of layers.

In Example 51, the subject matter of any one or more of Examples 44-50 optionally includes training the CNN, the training of the CNN comprising: executing the CNN using first training data; determining a model loss metric based on the executing of the CNN using the first training data; determining a model complexity metric based on the executing of the CNN using the first training data; and modifying at least one layer of the CNN, the modifying being based on the model loss metric and the model complexity metric.

In Example 52, the subject matter of any one or more of Examples 44-51 optionally includes responsive to the predicted sensor state indicating an error condition, terminating a sensor session of the continuous analyte sensor.

In Example 53, the subject matter of any one or more of Examples 44-52 optionally includes responsive to the predicted sensor state indicating an error condition, ceasing to provide a bias to the continuous analyte sensor.

Example 54 is a non-transitory machine-readable medium comprising instructions thereon that, when executed by at least one processor, cause the at least one processor to perform operations comprising: accessing sensor data generated by a continuous analyte sensor over a time period; executing a computerized model based on the sensor data, the computerized model comprising a convolutional neural network (CNN), the CNN comprising a plurality of layers; generating a predicted sensor state based on an output of the computerized model; and responsive to the predicted sensor state indicating an error condition, modifying a user interface output to suspend display of an output of the continuous analyte sensor.

In Example 55, the subject matter of Example 54 optionally includes the CNN comprising a plurality of layers, a first layer of the plurality of layers receiving an output of a first prior layer and an input to a second prior layer.

In Example 56, the subject matter of any one or more of Examples 54-55 optionally includes a first layer of the plurality of layers receiving an output of a first prior layer and an input to the first prior layer.

In Example 57, the subject matter of any one or more of Examples 54-56 optionally includes the operations further comprising training the CNN, the training of the CNN comprising: selecting a first set of dropout cells from at least one of the plurality of layers; deactivating the first set of dropout cells; executing the CNN using first training data, the CNN being configured with the first set of dropout cells deactivated during the executing; and modifying at least one layer of the CNN based on the executing of the CNN using the first training data.

In Example 58, the subject matter of Example 57 optionally includes the training of the CNN further comprising: re-activating the first set of dropout cells; selecting a second set of dropout cells from at least one of the plurality of layers; executing the CNN using second training data, the CNN being configured with the second set of dropout cells deactivated during the executing; and modifying at least one layer of the CNN based on the executing of the CNN using the second training data.

In Example 59, the subject matter of any one or more of Examples 57-58 optionally includes the deactivating of the first set of dropout cells comprising, for a first cell of the first set of dropout cells: modifying the CNN to remove an input to the first cell; and modifying the CNN to remove an output from the first cell.

In Example 60, the subject matter of any one or more of Examples 57-59 optionally includes the selecting of the first set of dropout cells comprising randomly or pseudo-randomly selecting a first cell from one of the plurality of layers.

In Example 61, the subject matter of any one or more of Examples 54-60 optionally includes the operations further comprising training the CNN, the training of the CNN comprising: executing the CNN using first training data; determining a model loss metric based on the executing of the CNN using the first training data; determining a model complexity metric based on the executing of the CNN using the first training data; and modifying at least one layer of the CNN, the modifying being based on the model loss metric and the model complexity metric.

In Example 62, the subject matter of any one or more of Examples 54-61 optionally includes the operations further comprising, responsive to the predicted sensor state indicating an error condition, terminating a sensor session of the continuous analyte sensor.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments described in the present document.

FIG. 1 is a diagram showing one example of an environment including an analyte sensor system.

FIG. 2 is a diagram showing one example of a medical device system including the analyte sensor system of FIG. 1.

FIG. 3 is an illustration of an example analyte sensor.

FIG. 4 is an illustration of another example analyte sensor.

FIG. 5 is an enlarged view of an example analyte sensor portion.

FIG. 6 is a cross-sectional view of the analyte sensor of FIGS. 3 and 4.

FIG. 7 is a schematic illustration of a circuit that represents the behavior of an example analyte sensor.

FIG. 8 is a diagram showing one example of an analyte sensor system including an analyte sensor and sensor electronics.

FIG. 9 is a diagram showing an example plot of sensor sensitivities of a group of continuous glucose sensors as a function of time during a sensor session.

FIG. 10 is a diagram showing three example plots of conversion functions at three different time periods of a sensor session.

FIG. 11 is a diagram showing one example of an analyte sensor system.

FIG. 12 is a flowchart showing one example of a process flow that may be executed in an analyte sensor system to detect and respond to potential sensor fault.

FIG. 13 is a flowchart showing one example of a process flow that may be executed to deploy an analyte sensor system.

FIG. 14 is a diagram showing an example architecture of a trained computerized model for determining whether an analyte sensor is in an end-of-life state or other error condition.

FIG. 15 is a diagram 1500 showing a portion of a computerized model including Convolutional Neural Network (CNN) layers.

FIG. 16 is a flowchart showing one example of a process flow that may be executed to train a computerized model utilizing dropout.

FIG. 17 is a flowchart showing one example of a process flow that may be executed train a computerized model considering model loss as well as complexity.

FIG. 18 is a block diagram illustrating a computing device hardware architecture, within which a set or sequence of instructions can be executed to cause a machine to perform examples of any one of the methodologies discussed herein.

### DETAILED DESCRIPTION

Various examples described herein are directed to analyte sensor systems and methods for using analyte sensor systems. An analyte sensor system includes an analyte sensor that is placed in contact with a bodily fluid of a host to measure a concentration of an analyte, such as glucose, in the bodily fluid. In some examples, the analyte sensor is inserted into the host to contact the bodily fluid in vivo. In some examples, the analyte sensor is inserted subcutaneously to contact interstitial fluid below the host's skin.

When the analyte sensor is exposed to analyte in the host's bodily fluid, an electrochemical reaction between the analyte sensor and the analyte causes the analyte sensor to generate a raw sensor signal that is indicative of the analyte concentration in the bodily fluid. For example, the analyte sensor may include two or more electrodes. Sensor electronics of the analyte sensor system apply a bias condition to the electrodes. The bias condition may be, for example, a potential difference applied between a working electrode of the analyte sensor and a reference electrode of the analyte sensor. The bias condition promotes the electrochemical reaction between the analyte and the analyte sensor, resulting in a current between the working electrode and at least one other analyte sensor electrode. The raw sensor signal may be and/or may be based on the current.

The sensor electronics uses the raw sensor signal to determine an estimated analyte value. In some examples, the sensor electronics are also programmed to output result data, which may include the estimated analyte value or other data. In some examples, the sensor electronics communicate result data to one or more other external devices.

In some examples, an analyte sensor has a useful life during which the sensor can provide reliable sensor data. After the useful life, the sensor may no longer be reliable. For example, after the useful life, the sensor may provide sensor data that less accurately correlates to the analyte concentration at the host. Various analyte sensor systems may be arranged to prevent use of an analyte sensor beyond the useful life of the analyte sensor. For example, an analyte sensor system may be configured to notify a user to cease using the analyte sensor (and change to a new analyte sensor) after it has been determined that the sensor has reached an end-of- life state and, therefore, should no longer be used.

Various methods can be used to determine whether a sensor has reached an end-of-life state. In some examples, a sensor system may determine that an end-of-life state has been reached when a predetermined amount of time has transpired since the sensor was first used (e.g., when first implanted into a host, when first electrically connected to sensor electronics, and/or the like). Also, in some examples, a sensor system is configured to detect a fault in the performance of the analyte sensor, where the detection of the fault is an indication that the sensor has reached an end-of-life state or is otherwise unsuitable for use.

In some embodiments, a continuous analyte sensor, such as a continuous glucose sensor, may exhibit a progressive decline in performance near the end of its useful life. The progressive decline in performance near the end of sensor life may be referred to as progressive sensor decline (PSD). In some examples, PSD may be severe enough to warrant discontinuing use of the sensor before one or more other end-of-life indicators, such as, for example the passage of the predetermined amount of time since the sensor was first used. Accordingly, in some examples, an analyte sensor system monitors the analyte sensor to identify indicators of PSD. When the analyte sensor system determines that the analyte sensor is exhibiting PSD, it may determine that the sensor is in an end-of-life state and should no longer be used.

Once it is determined that the sensor should no longer be used, the sensor system may execute a responsive action. The responsive action may be intended to prevent use of the sensor. In some examples, the responsive action may include notifying the host or other user by audibly and/or visually prompting the user to cease using the current sensor and/or begin using a new sensor. In some examples, the responsive action may include shutting down a display of the sensor system or ceasing to display new (or real-time) sensor data on the display. Also, in some examples, the responsive action may include ending a sensor session with the current analyte sensor. This can include, for example, ceasing to provide an electrical bias to the analyte sensor.

An analyte sensor system may determine whether an analyte sensor has entered an end-of-life state by monitoring properties of the sensor such as, for example, a noise level of the sensor signal, a drift in sensor sensitivity, and/or the like. In various examples, however, an analyte sensor inserted into a host can experience a number of environmental and/or conditions and may exhibit various responses that affect sensor performance. Some behaviors of the analyte sensor reflect temporary responses to environmental or use conditions, while other behaviors of the analyte sensor indicate that the sensor has entered an end-of-life state or has otherwise experienced a fault. Distinguishing between a temporary response to sensor conditions and an end-of-life or other fault state can be challenging.

Various examples address these and other challenges using a trained computerized model. The trained computerized model may be configured and trained to identify when a sensor has entered an end-of-life state or otherwise experienced a fault. In some examples, the trained computerized model includes a neural network having at least one Long Short-Term Memory (LSTM) element. The neural network, in some examples, comprises multiple LSTM layers operating on different time frames of the sensor signal.

For example, the trained computerized model may receive sensor data generated by the sensor over a time period. In some examples, the time period is from the most recent time that sensor data is received from the analyte sensor extending backwards in time. The neural network may be or include a hierarchal LSTM model comprising different LSTM layers that receive sensor data generated during different portions of the time period. For example, a first LSTM layer may receive sensor data captured over a previous 11 hours, a previous 12 hours, a previous 13 hours, and/or the like, a second LSTM layer may receive sensor data captured over a previous 5 minutes, a previous 10 minutes, a previous 20 minutes and/or the like, a third LSTM layer may receive sensor data captured over a previous 10 seconds, a previous 20 seconds, a previous 30 seconds, a previous 45 seconds, and/or the like, and so on. In this way, the trained computerized model may be configured to detect indicators of an end-of-life state or other fault over different time periods. In this way, the trained computerized model may capture indicators of an end-of-life state or other fault that may not otherwise be readily apparent.

In some examples the computerized model is or includes a convolutional neural network (CNN) trained to detect when an analyte sensor has entered an end-of-life state or otherwise experienced a fault indicating that the sensor is no longer suitable for use. In some examples, the CNN may be arranged with a residual connection. According to a residual connection, one or more cells of the neural network receive as input both the output of a previous layer cell and the input to the previous layer cell. The residual connection may improve the performance of the CNN and simplify training.

In some examples, the CNN may be trained in a manner that improves its performance in detecting an end-of-life state or other fault in a continuous analyte sensor. For example, the CNN may be trained in a plurality of epochs. In each epoch, the CNN model may receive labeled training data and generate a corresponding results. The results may be used to modify the various elements of the CNN, for example, by comparing the results to corresponding labels, for example, according to backpropagation or another suitable technique. Additional training epochs may be performed, for example, until the loss (e.g., based on the difference between the model output in the labels of the training data) meets a threshold, until a predetermined number of epochs have been performed, and/or the like.

In some examples, the CNN may be trained utilizing dropouts. For example, prior to one or more training epochs, a portion of the cells of the CNN may be deactivated. The deactivated cells may be referred to as dropout cells. In some examples, dropout cells for a particular training epoch may be selected randomly.

Deactivating a dropout cell may involve modifying the CNN to remove an input to the dropout cell and to remove an output of the dropout cell. For example, the input of a dropout cell may be provided as input to other cells in the CNN that were otherwise configured to receive an output of the dropout cell. A training epoch may be performed with one or more dropout cells removed from the CNN. At the conclusion of the training epoch, the dropout cells may be re-activated. In some examples, different sets of dropout cells may be selected for different training epochs. In some examples, the use of dropout cells during training of a trained computerized model may improve the performance of the model in detecting an end-of-life state or other analyte sensor fault. For example, the use of dropout cells reduce the likelihood that the trained computerized model will overfit to the training data.

Also, in some examples, the CNN may be trained utilizing weight regularization such as, for example, L2 weight regularization. According to weight regularization, the CNN may be trained in a number of training epochs. After the model is executed in each epoch, a model loss metric may be found. In addition to the model loss, a model complexity metric. Modifications to the trained computerized model after the training epoch may be determined to minimize both the model loss and the model complexity. This may improve the performance of the trained computerized model in detecting that an analyte sensor has entered an end- of-life state or other fault condition, for example, by reducing the likelihood that the trained computerized model will overfit to the training data.

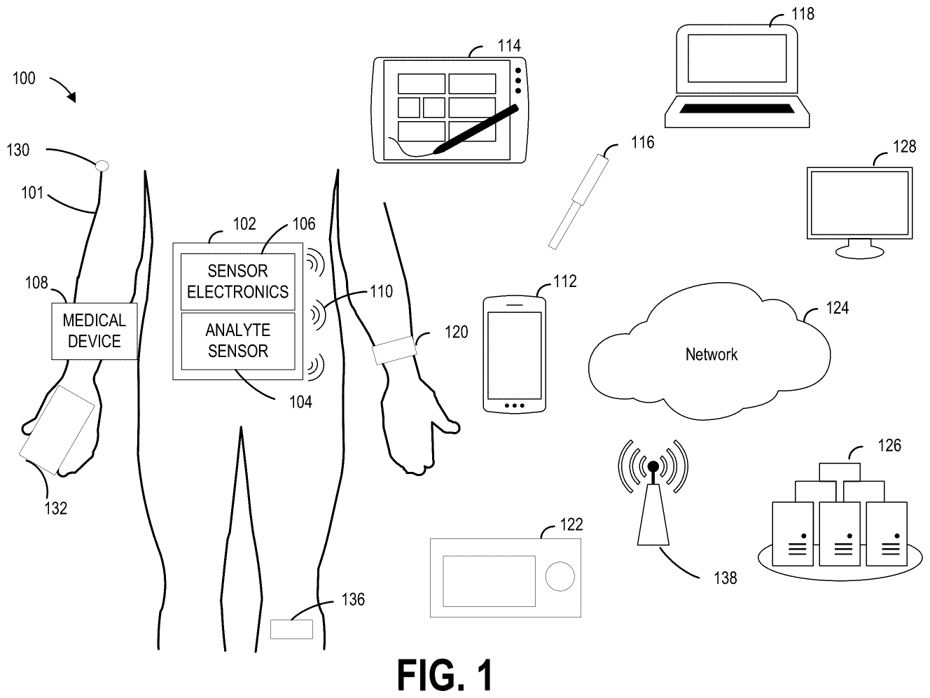

FIG. 1 is a diagram showing one example of an environment 100 including an analyte sensor system 102. The analyte sensor system 102 is coupled to a host 101, which may be a human patient. In some examples, the host is subject to a temporary or permanent diabetes condition or other health condition that makes analyte monitoring useful.

The analyte sensor system 102 includes an analyte sensor 104. In some examples, the analyte sensor 104 is or includes a glucose sensor configured to measure a glucose concentration in the host 101. The analyte sensor 104 can be exposed to analyte at the host 101 in any suitable way. In some examples, the analyte sensor 104 is fully implantable under the skin of the host 101. In other examples, the analyte sensor 104 is wearable on the body of the host 101 (e.g., on the body but not under the skin). Also, in some examples, the analyte sensor 104 is a transcutaneous device (e.g., with a sensor residing at least partially under or in the skin of a host). It should be understood that the devices and methods described herein can be applied to any device capable of detecting a concentration of an analyte, such as glucose, and providing an output signal that represents the concentration of the analyte.

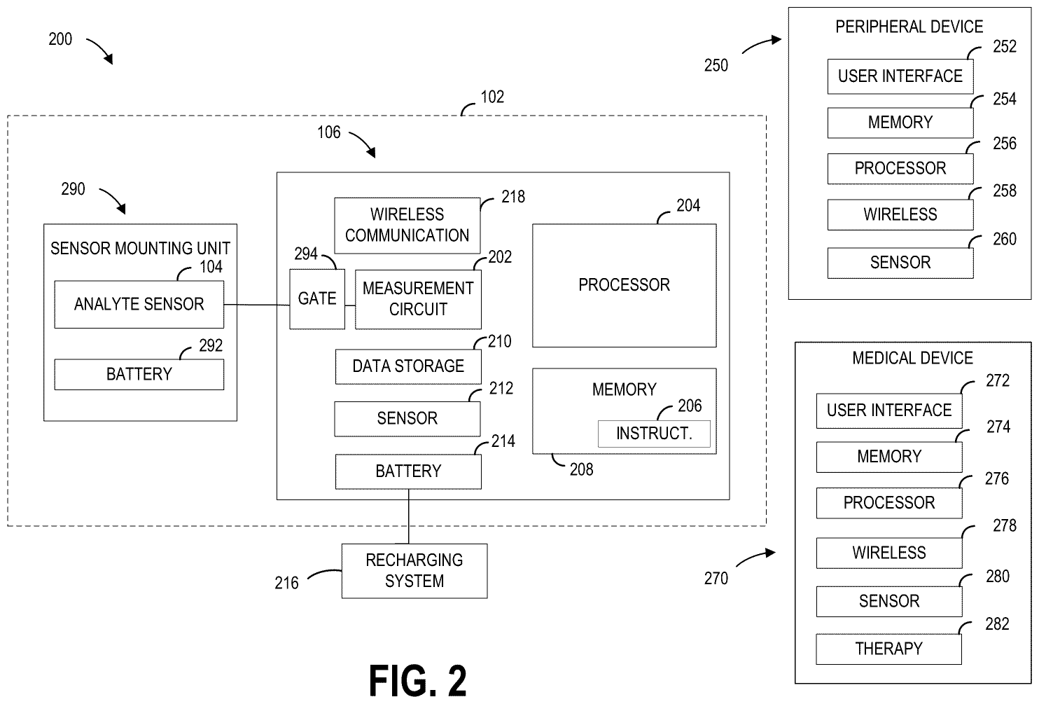

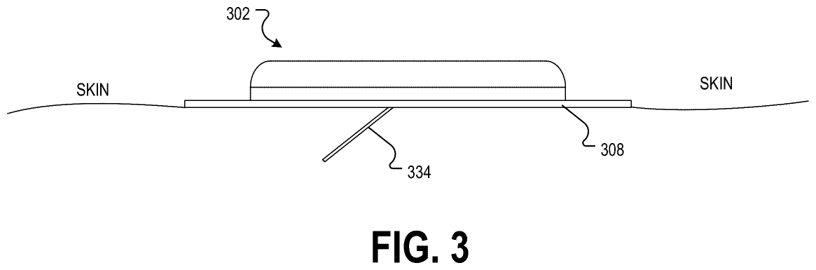

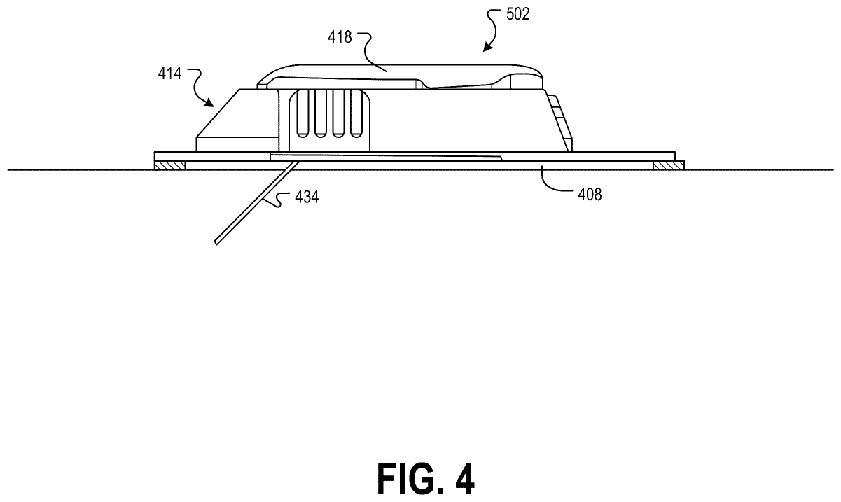

In the example of FIG. 1, the analyte sensor system 102 also includes sensor electronics 106. In some examples, the sensor electronics 106 and analyte sensor 104 are provided in a single integrated enclosure (See FIG. 3). In other examples, the analyte sensor 104 and sensor electronics 106 are provided as separate components or modules (See FIG. 4). For example, the analyte sensor system 102 may include a disposable (e.g., single-use) sensor mounting unit that may include the analyte sensor 104, a component for attaching the analyte sensor 104 to a host (e.g., an adhesive pad), and/or a mounting structure configured to receive a sensor electronics unit including some or all of the sensor electronics 106 shown in FIG. 2. The sensor electronics unit may be reusable.

The analyte sensor 104 may use any known method, including invasive, minimally-invasive, or non-invasive sensing techniques (e.g., optically excited fluorescence, microneedle, transdermal monitoring of glucose), to provide a raw sensor signal indicative of the concentration of the analyte in the host 101. The raw sensor signal may be converted into calibrated and/or filtered analyte concentration data used to provide a useful value of the analyte concentration (e.g., estimated blood glucose concentration level) to a user, such as the host or a caretaker (e.g., a parent, a relative, a guardian, a teacher, a doctor, a nurse, or any other individual that has an interest in the wellbeing of the host 101).

In some examples, the analyte sensor 104 is or includes a continuous glucose sensor. A continuous glucose sensor can be or include a subcutaneous, transdermal (e.g., transcutaneous), and/or intravascular device. In some examples, such a sensor or device may recurrently (e.g., periodically, or intermittently) analyze sensor data. The glucose sensor may use any method of glucose measurement, including enzymatic, chemical, physical, electrochemical, spectrophotometric, polarimetric, calorimetric, iontophoretic, radiometric, immunochemical, and the like. In various examples, the analyte sensor system 102 may be or include a continuous glucose monitor sensor available from DexCom™, (e.g., the DexCom G5™sensor, Dexcom G6™ sensor, the DexCom G7™ sensor, or any variation thereof), from Abbott™ (e.g., the Libre™ sensor), or from Medtronic™ (e.g., the Enlite™ sensor).

In some examples, analyte sensor 104 includes an implantable glucose sensor, such as described with reference to U.S. Pat. No. 6,001,067 and U.S. Patent Publication No. US-2005-0027463-A1, which are incorporated by reference. In some examples, analyte sensor 104 includes a transcutaneous glucose sensor, such as described with reference to U.S. Patent Publication No. US-2006-0020187-A1, which is incorporated by reference. In some examples, analyte sensor 104 may be configured to be implanted in a host vessel or extracorporeally, such as is described in U.S. Patent Publication No. US-2007-0027385-A1, co-pending U.S. Patent Publication No. US-2008-0119703-A1 filed Oct. 4, 2006, U.S. Patent Publication No. US-2008-0108942-A1 filed on Mar. 26, 2007, and U.S. Patent Application No. US-2007-0197890-A1 filed on Feb. 14, 2007, all of which are incorporated by reference. In some examples, the continuous glucose sensor may include a transcutaneous sensor such as described in U.S. Pat. No. 6,565,509 to Say et al., which is incorporated by reference. In some examples, analyte sensor 104 may include a continuous glucose sensor that includes a subcutaneous sensor such as described with reference to U.S. Pat. No. 6,579,690 to Bonnecaze et al. or U.S. Pat. No. 6,484,046 to Say et al., which are incorporated by reference. In some examples, the continuous glucose sensor may include a refillable subcutaneous sensor such as described with reference to U.S. Pat. No. 6,512,939 to Colvin et al., which is incorporated by reference. The continuous glucose sensor may include an intravascular sensor such as described with reference to U.S. Pat. No. 6,477,395 to Schulman et al., which is incorporated by reference. The continuous glucose sensor may include an intravascular sensor such as described with reference to U.S. Pat. No. 6,424,847 to Mastrototaro et al., which is incorporated by reference.

The environment 100 may also include various other external devices including, for example, a medical device 108. The medical device 108 may be or include a drug delivery device such as an insulin pump or an insulin pen. In some examples, the medical device 108 includes one or more sensors, such as another analyte sensor, a heart rate sensor, a respiration sensor, a motion sensor (e.g., accelerometer), posture sensor (e.g., 3-axis accelerometer), acoustic sensor (e.g., to capture ambient sound or sounds inside the body). The medical device 108 may be wearable, e.g., on a watch, glasses, contact lens, patch, wristband, ankle band, or another wearable item, or may be incorporated into a handheld device (e.g., a smartphone). In some examples, the medical device 108 includes a multi-sensor patch that may, for example, detect one or more of an analyte levels (e.g., glucose, lactate, insulin, or other substance), heart rate, respiration (e.g., using impedance), activity (e.g., using an accelerometer), posture (e.g., using an accelerometer), galvanic skin response, tissue fluid levels (e.g., using impedance or pressure).

In some examples, the analyte sensor system 102 and the medical device 108 communicate with one another. Communication between the analyte sensor system 102 and medical device 108 may occur over any suitable wired connection and/or via a wireless communication signal 110. For example, the analyte sensor system 102 (e.g., the sensor electronics 106 thereof) may be configured to establish a communication connection with the medical device 108 using a suitable short-range communications medium such as, for example, a radio frequency medium (e.g., Bluetooth, Medical Implant Communication System (MICS), Wi-Fi, near field communication (NFC), radio frequency identification (RFID), Zigbee, Z-Wave or other communication protocols), an optical medium (e.g., infrared), a sonic medium (e.g., ultrasonic), a cellular protocol-based medium (e.g., Code Division Multiple Access (CDMA) or Global System for Mobiles (GSM)), and/or the like.

In some examples, the environment 100 also includes other external devices such as, for example, a wearable sensor 130. The wearable sensor 130 can include a sensor circuit (e.g., a sensor circuit configured to detect a glucose concentration or other analyte concentration) and a communication circuit, which may, for example, be an NFC circuit. In some examples, information from the wearable sensor 130 may be retrieved from the wearable sensor 130 using a user computing device 132, such as a smart phone, that is configured to communicate with the wearable sensor 130 via the wearable sensor's communication circuit, for example, when the user device 132 is placed near the wearable sensor 130. For example, swiping the user device 132 over the sensor 130 may retrieve sensor data from the wearable sensor 130 using NFC or other suitable wireless communication.

The use of NFC communication may reduce power consumption by the wearable sensor 130, which may reduce the size of a power source (e.g., battery or capacitor) in the wearable sensor 130 or extend the usable life of the power source. In some examples, the wearable sensor 130 may be wearable on an upper arm as shown. In some examples, a wearable sensor 130 may additionally or alternatively be on the upper torso of the patient (e.g., over the heart or over a lung), which may, for example, facilitate detecting heart rate, respiration, or posture. A wearable sensor 136 may also be on the lower body (e.g., on a leg) or other part of the body (e.g., on the abdomen).

In some examples, an array or network of sensors may be associated with the patient. For example, one or more of the analyte sensor system 102, and/or external devices, such as the medical device 108, wearable device 120 such as a watch, an additional wearable sensor 130 and/or the like, may communicate with one another via a short-range communication medium (e.g., Bluetooth, MICS, NFC, or any of the other options described above,). The additional wearable sensor 130 may be any of the examples described above with respect to medical device 108. The analyte sensor system 102, medical device 108, and additional sensor 130 on the host 101 are provided for illustration and description and are not necessarily drawn to scale.

The environment 100 may also include one or more other external devices such as a hand-held smart device 112 (e.g., smart phone), tablet 114, smart pen 116 (e.g., insulin delivery pen with processing and communication capability), computer 118, a wearable device 120 such as a watch, or peripheral medical device 122 (which may be a proprietary device such as a proprietary user device available from DexCom™), any of which may communicate with the analyte sensor system 102 via a short-range communication medium, such as indicated by wireless communication signal 110, and may also communicate over a network 124 with a server system (e.g., remote data center) or with a remote terminal 128 to facilitate communication with a remote user (not shown) such as a technical support staff member or a clinician.

The wearable device 120 may include an activity sensor, a heart rate monitor (e.g., light-based sensor or electrode-based sensor), a respiration sensor (e.g., acoustic-or electrode-based), a location sensor (e.g., GPS), or other sensors.

In some examples, the environment 100 includes a server system 126. The server system 126 can include one or more computing devices, such as one or more server computing devices. In some examples, the server system 126 is used to collect analyte data from the analyte sensor system 102 and/or analyte or other data from the plurality of other devices, and to perform analytics on collected data, generate, or apply universal or individualized models for glucose levels, and communicate such analytics, models, or information based thereon back to one or more of the devices in the environment 100. In some examples, the server system 126 gathers inter-host and/or intra-host break-in data to generate one or more break-in characteristics, as described herein.

The environment 100 may also include a wireless access point (WAP) 138 used to communicatively couple one or more of analyte sensor system 102, network 124, server system 126, medical device 108 or any of the peripheral devices described above. For example, WAP 138 may provide Wi-Fi and/or cellular connectivity within environment 100. Other communication protocols, such as NFC or Bluetooth, may also be used among devices of the environment 100.

FIG. 2 is a diagram showing one example of a medical device system 200 including the analyte sensor system 102 of FIG. 1. In the example of FIG. 2, the analyte sensor system 102 includes sensor electronics 106 and an example sensor mounting unit 290, although in some examples, it will be appreciated that the analyte sensor 104 and sensor electronics 106 may be included in a common enclosure. While a specific example of division of components between the sensor mounting unit 290 and sensor electronics 106 is shown, it is understood that some examples may include additional components in the sensor mounting unit 290 or in the sensor electronics 106, and that some of the components (e.g., a battery or supercapacitor) that are shown in the sensor electronics 106 may be alternatively or additionally (e.g., redundantly) provided in the sensor mounting unit 290.

In the example shown in FIG. 2, the sensor mounting unit 290 includes the analyte sensor 104 and a battery 292. In some examples, the sensor mounting unit 290 may be replaceable, and the sensor electronics 106 may include a debouncing circuit (e.g., gate with hysteresis or delay) to avoid, for example, recurrent execution of a power-up or power down process when a battery is repeatedly connected and disconnected or avoid processing of noise signal associated with removal or replacement of a battery.

The sensor electronics 106 may include electronics components that are configured to process sensor information, such as raw sensor signals, and generate corresponding analyte concentration values. The sensor electronics 106 may, for example, include electronic circuitry associated with measuring, processing, storing, or communicating continuous analyte sensor data, including prospective algorithms associated with processing and calibration of the raw sensor signal. The sensor electronics 106 may include hardware, firmware, and/or software that enables measurement of levels of the analyte via a glucose sensor. Electronic components may be affixed to a printed circuit board (PCB), or the like, and can take a variety of forms. For example, the electronic components may take the form of an integrated circuit (IC), such as an Application-Specific Integrated Circuit (ASIC), a microcontroller, and/or a processor.

In the example of FIG. 2, the sensor electronics 106 include a measurement circuit 202 (e.g., potentiostat) coupled to the analyte sensor 104 and configured to recurrently obtain analyte sensor readings using the analyte sensor 104. For example, the measurement circuit 202 may continuously or recurrently measure a raw sensor signal indicating a current flow at the analyte sensor 104 between a working electrode and a reference electrode. The sensor electronics 106 may include a gate circuit 294, which may be used to gate the connection between the measurement circuit 202 and the analyte sensor 104. For example, the analyte sensor 104 may accumulate charge over an accumulation period. After the accumulation period, the gate circuit 294 is opened so that the measurement circuit 202 can measure the accumulated charge. Gating the analyte sensor 104 may improve the performance of the sensor system 102 by creating a larger signal to noise or interference ratio (e.g., because charge accumulates from an analyte reaction, but sources of interference, such as the presence of acetaminophen near a glucose sensor, do not accumulate, or accumulate less than the charge from the analyte reaction).

The sensor electronics 106 may also include a processor 204. The processor 204 is configured to retrieve instructions 206 from memory 208 and execute the instructions 206 to control various operations in the analyte sensor system 102. For example, the processor 204 may be programmed to control application of bias potentials to the analyte sensor 104 via a potentiostat at the measurement circuit 202, interpret raw sensor signals from the analyte sensor 104, and/or compensate for environmental factors.

The processor 204 may also save information in data storage memory 210 or retrieve information from data storage memory 210. In various examples, data storage memory 210 may be integrated with memory 208, or may be a separate memory circuit, such as a non-volatile memory circuit (e.g., flash RAM). Examples of systems and methods for processing sensor analyte data are described in more detail herein and in U.S. Pat. Nos. 7,310,544 and 6,931,327.

The sensor electronics 106 may also include one or more sensors, such as the sensor 212, which may be coupled to the processor 204. The sensor 212 may be a temperature sensor, accelerometer, or another suitable sensor. The sensor electronics 106 may also include a power source such as a capacitor or battery 214, which may be integrated into the sensor electronics 106, or may be removable, or part of a separate electronics unit. The battery 214 (or other power storage component, e.g., capacitor) may optionally be rechargeable via a wired or wireless (e.g., inductive or ultrasound) recharging system 216. The recharging system 216 may harvest energy or may receive energy from an external source or on-board source. In various examples, the recharge circuit may include a triboelectric charging circuit, a piezoelectric charging circuit, an RF charging circuit, a light charging circuit, an ultrasonic charging circuit, a heat charging circuit, a heat harvesting circuit, or a circuit that harvests energy from the communication circuit. In some examples, the recharging circuit may recharge the rechargeable battery using power supplied from a replaceable battery (e.g., a battery supplied with a base component).

The sensor electronics 106 may also include one or more supercapacitors in the sensor electronics unit (as shown), or in the sensor mounting unit 290. For example, the supercapacitor may allow energy to be drawn from the battery 214 in a highly consistent manner to extend the life of the battery 214. The battery 214 may recharge the supercapacitor after the supercapacitor delivers energy to the communication circuit or to the processor 204, so that the supercapacitor is prepared for delivery of energy during a subsequent high-load period. In some examples, the supercapacitor may be configured in parallel with the battery 214. A device may be configured to preferentially draw energy from the supercapacitor, as opposed to the battery 214. In some examples, a supercapacitor may be configured to receive energy from a rechargeable battery for short-term storage and transfer energy to the rechargeable battery for long-term storage.

The supercapacitor may extend an operational life of the battery 214 by reducing the strain on the battery 214 during the high-load period. In some examples, a supercapacitor removes at least 10% of the strain off the battery during high-load events. In some examples, a supercapacitor removes at least 20% of the strain off the battery during high-load events. In some examples, a supercapacitor removes at least 30% of the strain off the battery during high-load events. In some examples, a supercapacitor removes at least 50% of the strain off the battery during high-load events.

The sensor electronics 106 may also include a wireless communication circuit 218, which may for example include a wireless transceiver operatively coupled to an antenna. The wireless communication circuit 218 may be operatively coupled to the processor 204 and may be configured to wirelessly communicate with one or more peripheral devices or other medical devices, such as an insulin pump or smart insulin pen.

In the example of FIG. 2, the medical device system 200 also includes optional external devices including, for example, a peripheral device 250. The peripheral device 250 may be any suitable user computing device such as, for example, a wearable device (e.g., activity monitor), such as a wearable device 120. In other examples, the peripheral device 250 may be a hand-held smart device (e.g., smartphone or other device such as a proprietary handheld device available from Dexcom), a tablet 114, a smart pen 116, or special-purpose computer 118 shown in FIG. 1.

The peripheral device 250 may include a user interface (UI) 252, a memory circuit 254, a processor 256, a wireless communication circuit 258, a sensor 260, or any combination thereof. The peripheral device 250 may not necessarily include all the components shown in FIG. 2. The peripheral device 250 may also include a power source, such as a battery.

The UI 252 may, for example, be provided using any suitable input/output device or devices of the peripheral device 250 such as, for example, a touch-screen interface, a microphone (e.g., to receive voice commands), or a speaker, a vibration circuit, or any combination thereof. The UI 252 may receive information from the host or another user (e.g., instructions, glucose values). The UI 252 may also deliver information to the host or other user, for example, by displaying UI elements at the UI 252. For example, UI elements can indicate glucose or other analyte concentration values, glucose or other analyte trends, glucose, or other analyte alerts, etc. Trends can be indicated by UI elements such as arrows, graphs, charts, etc.

The processor 256 may be configured to present information to a user, or receive input from a user, via the UI 252. The processor 256 may also be configured to store and retrieve information, such as communication information (e.g., pairing information or data center access information), user information, sensor data or trends, or other information in the memory circuit 254. The wireless communication circuit 258 may include a transceiver and antenna configured to communicate via a wireless protocol, such as any of the wireless protocols described herein. The sensor 260 may, for example, include an accelerometer, a temperature sensor, a location sensor, biometric sensor, or blood glucose sensor, blood pressure sensor, heart rate sensor, respiration sensor, or another physiologic sensor.

The peripheral device 250 may be configured to receive and display sensor information that may be transmitted by sensor electronics 106 (e.g., in a customized data package that is transmitted to the display devices based on their respective preferences). Sensor information (e.g., blood glucose concentration level) or an alert or notification (e.g., “high glucose level”, “low glucose level” or “fall rate alert” may be communicated via the UI 252 (e.g., via visual display, sound, or vibration). In some examples, the peripheral device 250 may be configured to display or otherwise communicate the sensor information as it is communicated from the sensor electronics 106 (e.g., in a data package that is transmitted to respective display devices). For example, the peripheral device 250 may transmit data that has been processed (e.g., an estimated analyte value level that may be determined by processing raw sensor data), so that a device that receives the data may not be required to further process the data to determine usable information (such as the estimated analyte value level). In other examples, the peripheral device 250 may process or interpret the received information (e.g., to declare an alert based on glucose values or a glucose trend). In various examples, the peripheral device 250 may receive information directly from sensor electronics 106, or over a network (e.g., via a cellular or Wi-Fi network that receives information from the sensor electronics 106 or from a device that is communicatively coupled to the sensor electronics 106).

In the example of FIG. 2, the medical device system 200 includes an optional medical device 270. For example, the medical device 270 may be an external device used in addition to or instead of the peripheral device 250. The medical device 270 may be or include any suitable type of medical or other computing device including, for example, the medical device 108, peripheral medical device 122, wearable device 120, wearable sensor 130, or wearable sensor 136 shown in FIG. 1. The medical device 270 may include a UI 272, a memory circuit 274, a processor 276, a wireless communication circuit 278, a sensor 280, a therapy circuit 282, or any combination thereof.

Similar to the UI 252, the UI 272 may be provided using any suitable input/output device or devices of the medical device 270 such as, for example, a touch-screen interface, a microphone, or a speaker, a vibration circuit, or any combination thereof. The UI 272 may receive information from the host or another user (e.g., glucose values, alert preferences, calibration coding). The UI 272 may also deliver information to the host or other user, for example, by displaying UI elements at the UI 252. For example, UI elements can indicate glucose or other analyte concentration values, glucose or other analyte trends, glucose, or other analyte alerts, etc. Trends can be indicated by UI elements such as arrows, graphs, charts, etc.

The processor 276 may be configured to present information to a user, or receive input from a user, via the UI 272. The processor 276 may also be configured to store and retrieve information, such as communication information (e.g., pairing information or data center access information), user information, sensor data or trends, or other information in the memory circuit 274. The wireless communication circuit 278 may include a transceiver and antenna configured to communicate via a wireless protocol, such as any of the wireless protocols described herein.

The sensor 280 may, for example, include an accelerometer, a temperature sensor, a location sensor, biometric sensor, or blood glucose sensor, blood pressure sensor, heart rate sensor, respiration sensor, or another physiologic sensor. The medical device 270 may include two or more sensors (or memories or other components), even though only one sensor 280 is shown in the example in FIG. 2. In various examples, the medical device 270 may be a smart handheld glucose sensor (e.g., blood glucose meter), drug pump (e.g., insulin pump), or other physiologic sensor device, therapy device, or combination thereof.

In examples where medical device 270 is or includes an insulin pump, the pump and the analyte sensor system 102 may be in two-way communication (e.g., so the pump can request a change to an analyte transmission protocol, e.g., request a data point or request data on a more frequent schedule), or the pump and analyte sensor system 102 may communicate using one-way communication (e.g., the pump may receive analyte concentration level information from the analyte sensor system). In one-way communication, a glucose value may be incorporated in an advertisement message, which may be encrypted with a previously shared key. In a two-way communication, a pump may request a value, which the analyte sensor system 102 may share, or obtain and share, in response to the request from the pump, and any or all of these communications may be encrypted using one or more previously shared keys. An insulin pump may receive and track analyte (e.g., glucose) values transmitted from analyte sensor system 102 using one-way communication to the pump for one or more of a variety of reasons. For example, an insulin pump may suspend or activate insulin administration based on a glucose value being below or above a threshold value.

In some examples, the medical device system 200 includes two or more peripheral devices and/or medical devices that each receive information directly or indirectly from the analyte sensor system 102. Because different display devices provide many different user interfaces, the content of the data packages (e.g., amount, format, and/or type of data to be displayed, alarms, and the like) may be customized (e.g., programmed differently by the manufacturer and/or by an end user) for each device. For example, referring now to the example of FIG. 1, a plurality of different peripheral devices may be in direct wireless communication with sensor electronics 106 (e.g., such as an on-skin sensor electronics 106 that are physically connected to the continuous analyte sensor 104) during a sensor session to enable a plurality of different types and/or levels of display and/or functionality associated with the displayable sensor information, or, to save battery power in the analyte sensor system 102, one or more specified devices may communicate with the analyte sensor system 102 and relay (i.e., share) information to other devices directly or through a server system (e.g., a network-connected data center) 126.

FIG. 3 is a side view of an example analyte sensor 334 that may be implanted into a host. An enclosure 302 may be adhered to the host's skin using an adhesive pad 308. The adhesive pad 308 may be formed from an extensible material, which may be removably attached to the skin using an adhesive. Sensor electronics may be positioned within the enclosure 302. The sensor 334 may extend from the enclosure 302 and under the skin of a host, as shown.

FIG. 4 is a side view of another example analyte sensor 434 in an arrangement including a mounting unit 414 and an electronics unit 418. The mounting unit 414 may be adhered to the host's skin using an adhesive pad 408, which may be like the adhesive pad 308 described herein. The electronics unit 418 comprises an enclosure 402 that may have sensor electronics positioned thereon. In some examples, the electronics unit 418 and mounting unit 414 are arranged in a manner like the sensor electronics 106 and sensor mounting unit 290 shown in FIGS. 1 and 2. For example, the sensor 434 may extend from the enclosure 402 via the mounting unit 414.

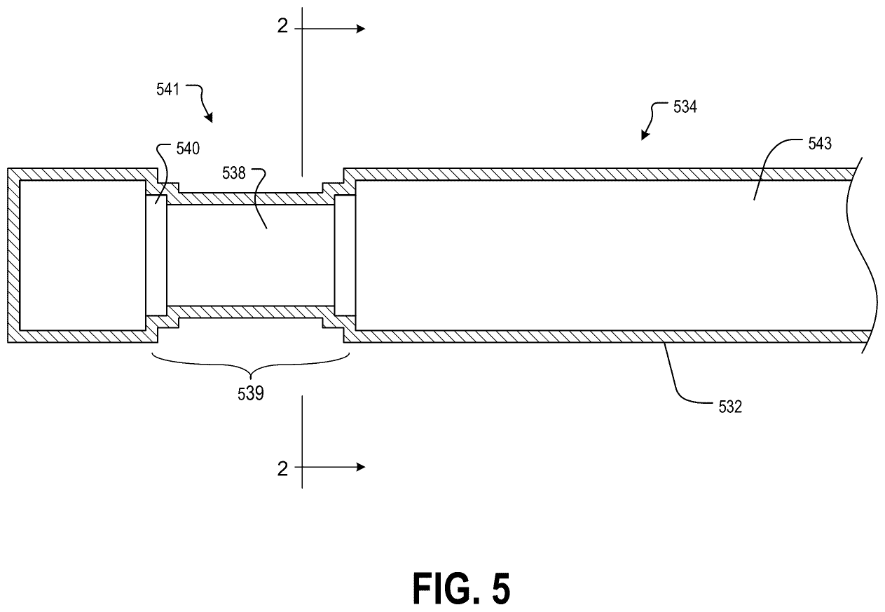

FIG. 5 is an enlarged view of a distal portion of an analyte sensor 534. The analyte sensor 534 illustrates one example arrangement that may be used to implement the analyte sensors described herein, such as, for example, the analyte sensors 104, 334, 434. The analyte sensor 534 may be adapted for insertion under the host's skin and may be mechanically coupled to an enclosure, such as the enclosures 402, and/or to a mounting unit 414. —The analyte sensor 534 may be electrically coupled to sensor electronics, which may be positioned within the enclosure 302, 402.

The example analyte sensor 534 shown in FIG. 5 includes an elongated conductive body 541. The elongated conductive body 541 can include a core with various layers positioned thereon. A first layer 538 that at least partially surrounds the core and includes a working electrode, for example located in window 539). In some examples, the core and the first layer 538 are made of a single material (such as, for example, platinum). In some examples, the elongated conductive body 541 is a composite of two conductive materials, or a composite of at least one conductive material and at least one non-conductive material. A membrane system 532 is located over the working electrode and may cover other layers and/or electrodes of the sensor 534, as described herein.

The first layer 538 may be formed of a conductive material. The working electrode (at window 539) is an exposed portion of the surface of the first layer 538. Accordingly, the first layer 538 is formed of a material configured to provide a suitable electroactive surface for the working electrode. Examples of suitable materials include, but are not limited to, platinum, platinum-iridium, gold, palladium, iridium, graphite, carbon, a conductive polymer, an alloy, and/or the like.

A second layer 540 surrounds at least a portion of the first layer 538, thereby defining boundaries of the working electrode. In some examples, the second layer 540 serves as an insulator and is formed of an insulating material, such as polyimide, polyurethane, parylene, or any other suitable insulating materials or materials. In some examples, the second layer 540 is configured such that the working electrode (of the layer 538) is exposed via the window 539.

In some examples, the sensor 534 further includes a third layer 543 comprising a conductive material. The third layer 543 may comprise a reference electrode. In some examples, the third layer 543, including the reference electrode, is formed of a silver-containing material that is applied onto the second layer 540 (e.g., an insulator). The silver-containing material may include various materials and be in various forms such as, for example, Ag/AgCl-polymer pasts, paints, polymer-based conducting mixtures, inks, etc.

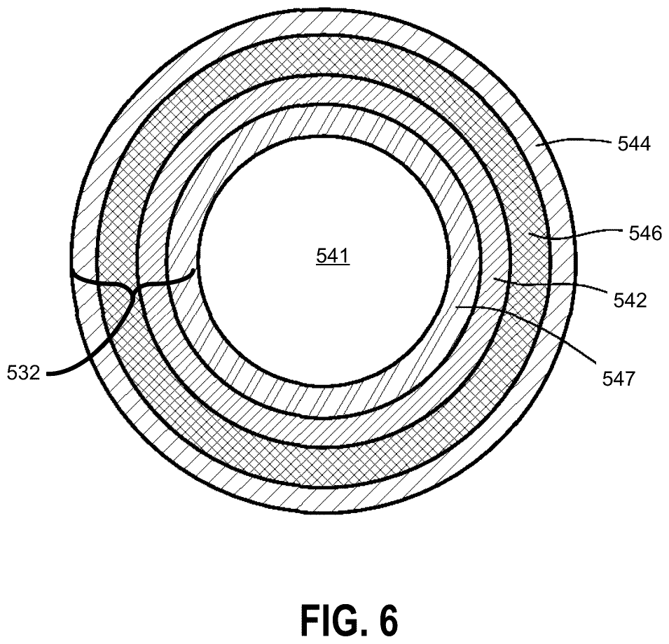

The analyte sensor 534 may include two (or more) electrodes, e.g., a working electrode at the layer 538 and exposed at window 539 and at least one additional electrode, such as a reference electrode of the layer 543. In the example arrangement of FIGS. 5-6, the reference electrode also functions as a counter electrode, although other arrangements can include a separate counter electrode. While the analyte sensor 534 may be used with a mounting unit in some examples, in other examples, the analyte sensor 534 may be used with other types of sensor systems. For example, the analyte sensor 534 may be part of a system that includes a battery and sensor in a single package, and may optionally include, for example, a near-field communication (NFC) circuit.

FIG. 6 is a cross-sectional view through the sensor 534 of FIG. 5 on plane 2-2 illustrating a membrane system 532. The membrane system 532 may include a number of domains (e.g., layers). In an example, the membrane system 532 may include an enzyme domain 542, a diffusion resistance domain 544, and a bioprotective domain 546 located around the working electrode. In some examples, a unitary diffusion resistance domain and bioprotective domain may be included in the membrane system 532 (e.g., wherein the functionality of both the diffusion resistance domain and bioprotective domain are incorporated into one domain).

The membrane system 532, in some examples, also includes an electrode layer 547. The electrode layer 547 may be arranged to provide an environment between the surfaces of the working electrode and the reference electrode that facilitates the electrochemical reaction between the electrodes. For example, the electrode layer 547 may include a coating that maintains a layer of water at the electrochemically reactive surfaces of the sensor 534.

In some examples, the sensor 534 may be configured for short-term implantation (e.g., from about 1 to 30 days). However, it is understood that the membrane system 532 can be modified for use in other devices, for example, by including only one or more of the domains, or additional domains. For example, a membrane system 532 may include a plurality of resistance layers, or a plurality of enzyme layers. In some examples, the resistance domain 544 may include a plurality of resistance layers, or the enzyme domain 542 may include a plurality of enzyme layers.

The diffusion resistance domain 544 may include a semipermeable membrane that controls the flux of oxygen and glucose to the underlying enzyme domain 542. As a result, the upper limit of linearity of glucose measurement is extended to a much higher value than that which is achieved without the diffusion resistance domain 544.

In some examples, the membrane system 532 may include a bioprotective domain 546, also referred to as a domain or bio-interface domain, comprising a base polymer. However, the membrane system 532 of some examples can also include a plurality of domains or layers including, for example, an electrode domain, an interference domain, or a cell disruptive domain, such as described in more detail elsewhere herein and in U.S. Pat. Nos. 7,494,465, 8,682,608, and 9,044,199, which are incorporated herein by reference in their entirety.

It is to be understood that sensing membranes modified for other sensors, for example, may include fewer or additional layers. For example, in some examples, the membrane system 532 may comprise one electrode layer, one enzyme layer, and two bioprotective layers, but in other examples, the membrane system 532 may comprise one electrode layer, two enzyme layers, and one bioprotective layer. In some examples, the bioprotective layer may be configured to function as the diffusion resistance domain 544 and control the flux of the analyte (e.g., glucose) to the underlying membrane layers.

In some examples, one or more domains of the sensing membranes may be formed from materials such as silicone, polytetrafluoroethylene, polyethylene-co-tetrafluoroethylene, polyolefin, polyester, polycarbonate, biostable polytetrafluoroethylene, homopolymers, copolymers, terpolymers of polyurethanes, polypropylene (PP), polyvinylchloride (PVC), polyvinylidene fluoride (PVDF), polybutylene terephthalate (PBT), polymethylmethacrylate (PMMA), polyether ether ketone (PEEK), polyurethanes, cellulosic polymers, poly (ethylene oxide), poly (propylene oxide) and copolymers and blends thereof, polysulfones and block copolymers thereof including, for example, di-block, tri-block, alternating, random and graft copolymers.

In some examples, the sensing membrane can be deposited on the electroactive surfaces of the electrode material using known thin or thick film techniques (for example, spraying, electro-depositing, dipping, or the like). The sensing membrane located over the working electrode does not have to have the same structure as the sensing membrane located over the reference electrode; for example, the enzyme domain 542 deposited over the working electrode does not necessarily need to be deposited over the reference or counter electrodes.

Although the examples illustrated in FIGS. 5-6 involve circumferentially extending membrane systems, the membranes described herein may be applied to any planar or non-planar surface, for example, the substrate-based sensor structure of U.S. Pat. No. 6,565,509 to Say et al., which is incorporated by reference.

In an example in which the analyte sensor 534 is a glucose sensor, glucose analyte can be detected utilizing glucose oxidase. Glucose oxidase reacts with glucose to produce hydrogen peroxide (H₂O₂). The hydrogen peroxide reacts with the surface of the working electrode, producing two protons (2H⁺), two electrons (2e⁻) and one molecule of oxygen (O₂). This produces an electronic current that may be detected by the sensor electronics 106. The amount of current is a function of the glucose concentration level. A calibration curve may be used to provide an estimated glucose concentration level based on a measured current. The amount of current may also be a function of the diffusivity of glucose through the sensor membrane. The glucose diffusivity may change over time, which may cause the sensor glucose sensitivity to change over time, or “drift.”

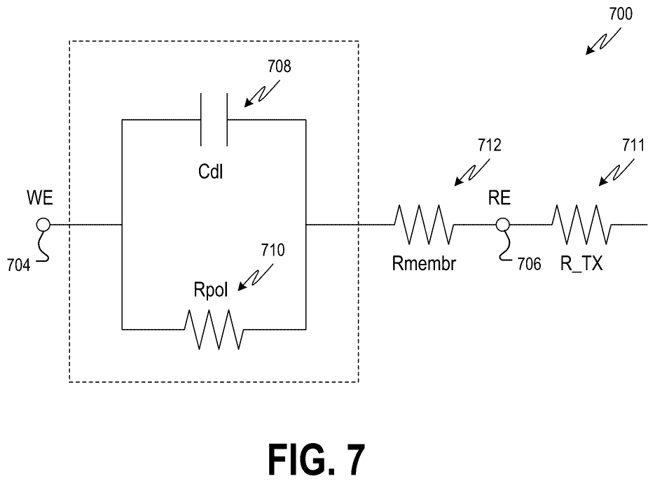

FIG. 7 is a schematic illustration of a circuit 700 that represents the behavior of an example analyte sensor, such as the analyte sensor 534 shown in FIGS. 5-6. As described above, the interaction of hydrogen peroxide (generated from the interaction between glucose analyte and glucose oxidase) and working electrode (WE) 704 produces a voltage differential between the working electrode (WE) 704 and reference electrode (RE) 706 which drives a current. The current may make up all or part of a raw sensor signal that is measured by sensor electronics, such as the sensor electronics 106 of FIGS. 1-2, and used to estimate an analyte concentration (e.g., glucose concentration).

The circuit 700 also includes a double-layer capacitance (Cdl) 708, which occurs at an interface between the working electrode (WE) 704 and the adjacent membrane (not shown in FIG. 7, see, e.g., FIGS. 5-6 above). The double-layer capacitance (Cdl) may occur at an interface between the working electrode 704 and the adjacent membrane due to the presence of two layers of ions with opposing polarity, as may occur during application of an applied voltage between the working electrode 704 and reference electrode 706. The equivalent circuit 700 may also include a polarization resistance (Rpol) 710, which may be relatively large, and may be modeled, for example, as a static value (e.g., 100 mega-Ohms), or as a variable quantity that varies as a function of glucose concentration level.

An estimated analyte value may be determined from a raw sensor signal based upon a measured current (or charge flow) through the analyte sensor membrane 712 when a bias potential is applied to the sensor circuit 700. For example, sensor electronics or another suitable computing device can use the raw sensor signal and a sensitivity of the sensor, which correlates a detected current flow to a glucose concentration level, to generate the estimated analyte value. In some examples, the device also uses a break-in characteristic, as described herein.

With reference to the equivalent circuit 700, when a voltage is applied across the working and reference electrodes 704 and 706, a current may be considered to flow (forward or backward depending on polarity) through the internal electronics of transmitter (represented by R_Tx_internal) 711; through the reference electrode (RE) 706 and working electrode (WE) 704, which may be designed to have a relatively low resistance; and through the sensor membrane 712 (Rmembr, which is relatively small). Depending on the state of the circuit, current may also flow through, or into, the relatively large polarization resistance 710 (which is indicated as a fixed resistance but may also be a variable resistance that varies with the body's glucose level, where a higher glucose level provides a smaller polarization resistance), or into the double-layer capacitance 708 (i.e., to charge the double-layer membrane capacitor formed at the working electrode 704), or both.

The impedance (or conductance) of the membrane (Rmembr) 712 is related to electrolyte mobility in the membrane, which is in turn related to glucose diffusivity in the membrane. As the impedance goes down (i.e., conductance goes up, as electrolyte mobility in the membrane 712 goes up), the glucose sensitivity goes up (i.e., a higher glucose sensitivity means that a particular glucose concentration will produce a larger signal in the form of more current or charge flow). Impedance, glucose diffusivity, and glucose sensitivity are further described in U.S. Patent Publication No. US2012/0262298, which is incorporated by reference in its entirety.

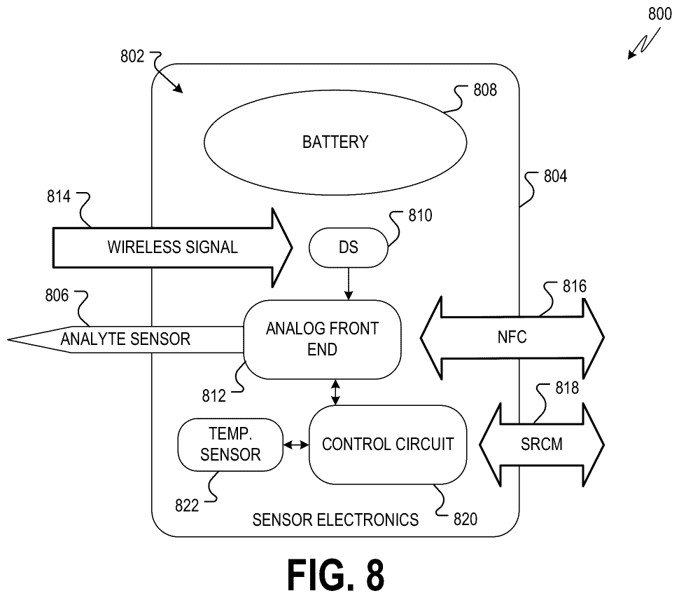

FIG. 8 is a diagram showing one example of an analyte sensor system 800 including an analyte sensor 806 and sensor electronics 802. The analyte sensor 806 may be arranged, for example, as described herein with respect to FIGS. 3-6 and/or according to any other suitable arrangement such as, for example a planar sensor arrangement.

The sensor electronics 802 are positioned within an enclosure 804. The analyte sensor 806 extends from the enclosure 804 as shown. In some examples, the analyte sensor 806 is mechanically coupled to the enclosure 804, for example, as illustrated in FIG. 3. In some examples, the analyte sensor 806 is mechanically coupled to a sensor mounting unit, as described herein.

The analyte sensor 806 may be in electrical communication with an analog front end 812 of the sensor electronics 802. In examples in which the analyte sensor 806 is mechanically coupled to the enclosure 804, the analyte sensor 806 may extend through the enclosure 804 in a sealed manner and be in direct electrical contact with the analog front end 812. In examples in which the analyte sensor 806 is mechanically coupled to a sensor mounting unit, an electrical connection between the analyte sensor 806 and the analog front end 812 may be via a connector between the sensor mounting unit and the enclosure 804.

The analog front end 812 may be configured to receive analog electrical signals from various components of the sensor electronics 802 and provide corresponding digital electrical signals to a sensor electronics 820. For example, as described, the analyte sensor 806 may be in electrical communication with the analog front end 812 to provide an analog raw sensor signal to the analog front end 812. The analog front end 812 may comprise various amplifiers, filters, conditioners, analog-to-digital converters, and/or the like to condition the raw sensor signal and covert it to a digital raw sensor signal, which may be provided to the sensor electronics 820. The sensor electronics 820 may be configured to convert the digital raw sensor signal to an estimated analyte value, which may be output as described herein.

The analog front end 812 may also be in communication with a deployment sensor 810. The deployment sensor 810 receives a wireless signal 814. The wireless signal 814 may be generated by a transmitter at an applicator. The deployment sensor 810 may be configured to detect the wireless signal 814 and generate an output indicative of the state of the wireless signal 814. In examples where the wireless signal 814 is a magnetic signal, the deployment sensor 810 may be or comprise a magnetic sensor, such as a Tunnel Magento-Resistance effect (TMR) sensor. In examples where the wireless signal 814 is an optical signal, the deployment sensor 810 may be or comprise a photoresistor or another optical sensor. In examples where the wireless signal 814 is a radio frequency (RF) or other similar signal, the deployment sensor 810 may comprise an antenna and/or other RF receiver components. The analog front end 812 may receive an output of the deployment sensor 810 and provide the output to the sensor electronics 820 for further processing. In some examples, as shown in FIG. 8, the analog front end 812 and sensor electronics 820 may be coupled to the analyte sensor 806 prior to deployment. In this way, the analog front end 812 and sensor electronics 820 may detect deployment of the analyte sensor 806 (e.g., via wireless signal 814) close in time to the actual insertion of the analyte sensor 806.

In the example of FIG. 8, the analog front end 812 comprises circuitry for receiving and transmitting near field communication (NFC) signals 816. NFC signals 816 may be utilized to communicate with various external devices. In some examples, the wireless signal 814 may be implemented as an NFC signal 816.

The sensor electronics 820 is configured to receive digital signals from the analog front end 812 and perform various processing on the signals. For example, the sensor electronics 820 may be and/or include a microcontroller or other processor. One or more processers of the sensor electronics 820 may be programmed to execute software instructions for executing various operations, for example, as described herein. The software instructions may be stored at a data storage that may be part of the sensor electronics 820 or may be implemented at a different location.

In some examples, the sensor electronics 820 converts a raw sensor signal (e.g., a digital raw sensor signal) to a corresponding estimated analyte value. The sensor electronics 820 may provide result data including the estimated analyte value as an output, for example, in the manner described herein. In the example of FIG. 8, the sensor electronics 820 is in communication with a temperature sensor 822, for example, as described herein. In this example, the temperature sensor 822 is a digital sensor. In other examples, an analog temperature sensor (or other sensor, as described herein) may be in communication with the sensor electronics 820 via the analog front end 812. In some examples, the sensor electronics 820 is also configured to communicate with one or more external devices via a short-range wireless communication medium 818 such as, for example, Bluetooth®, Bluetooth LE® and/or the like.

The sensor electronics 820 may also be programmed to transition the analyte sensor system 800 from a sleep mode to an active mode. For example, various components of the sensor electronics 802 are powered by a battery 808. In some examples, the battery 808 is installed at the time that the analyte sensor system 800 is manufactured. In some examples, the analyte sensor system 800 is configured to operate for its full lifecycle on a single charge of the battery 808. Accordingly, the analyte sensor system 800 may be configured in a sleep mode after manufacturing. In the sleep mode, the sensor electronics 802 maintains the analyte sensor system 800 in an arrangement for low power consumption. For example, the non-essential components of the analyte sensor system 800 may be switched off. Also, for example, operations executed by the sensor electronics 802 may be minimized to save power. The sensor electronics 820 may be programmed to maintain the sleep mode until the analyte sensor 806 is inserted in vivo into a host and a sensor session is to begin.

When the sensor electronics 820 determines that a sensor session is to begin, the sensor electronics 820 transitions the analyte sensor system 800 into the active mode. This may include, for example, applying a bias condition to the analyte sensor, using the raw sensor signal (e.g., a digital raw sensor signal) to determine and output an estimated analyte concentration, and/or the like. The sensor electronics 802 may determine to transition the analyte sensor system 800 to the active mode, for example, using the wireless signal 814 and/or the raw sensor signal provided by the analyte sensor 806. Although specific circuitry, components, and interconnections are illustrated in the example analyte sensor system 800 shown in FIG. 8, alternative examples may incorporate additional, fewer, and/or alternative circuitry, components, and/or interconnections.

### Determination of Sensor Sensitivity

As described elsewhere herein, in certain examples, self-calibration of the analyte sensor system can be performed by determining sensor sensitivity based on a sensitivity profile (and a measured or estimated baseline), so that Equation [1] can be solved:

y=mx+b [1]

In Equation [1], y represents the sensor signal, x represents the estimated glucose concentration (mg/dL), m represents the sensor sensitivity to the analyte (counts/mg/dL), and b represents the baseline signal. The sensor signal y and baseline b are based on a current generated by the analyte sensor. For example, the sensor signal y and baseline b may be expressed in Amps (e.g. milliamps or microamps) or in counts generated by a current sensor of the sensor electronics. In some examples, Equation [1] can be used to form a conversion function for converting the sensor signal into an estimated glucose or other analyte concentration.

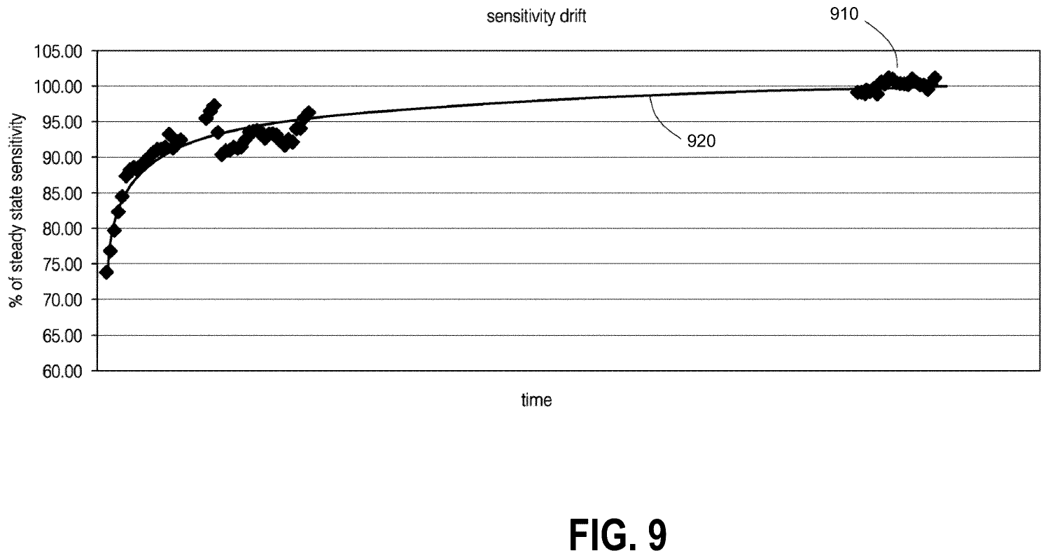

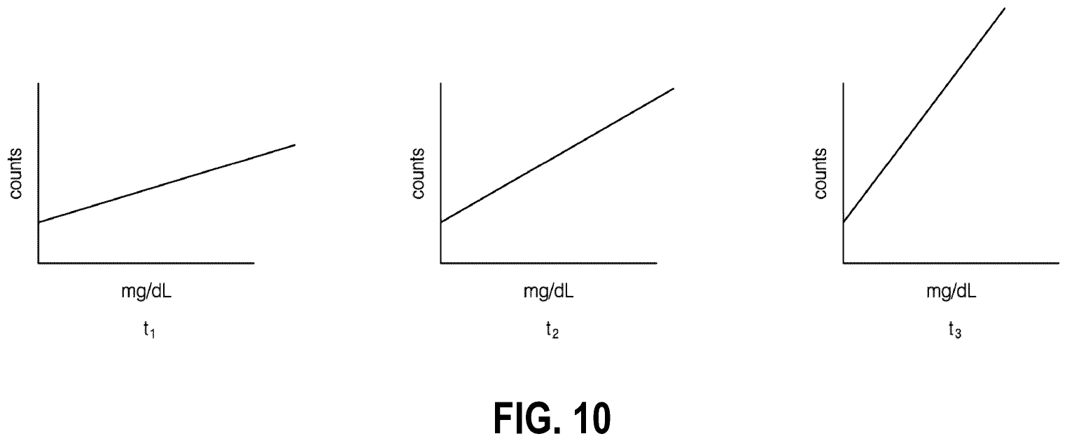

In some examples, it has been found that a sensor's sensitivity to analyte concentration during a sensor session will change or drift as a function of time. FIG. 9 illustrates this phenomenon and provides a plot of sensor sensitivities 910 of a group of continuous glucose sensors as a function of time during a sensor session. FIG. 10 provides three plots of conversion functions at three different time periods of a sensor session. As shown in FIG. 10, the three conversion functions have different slopes, each of which correspond to a different sensor sensitivity. Accordingly, the differences in slopes over time illustrate that changes or drift in sensor sensitivity occur over a sensor session.

Referring back to the study associated with FIG. 9, the sensors were made in substantially the same way under substantially the same conditions. The sensor sensitivities associated with the y-axis of the plot are expressed as a percentage of a substantially steady state sensitivity that was reached about three days after start of the sensor session. In addition, these sensor sensitivities correspond to measurements obtained from analyte concentration tests performed using a glucometer or other suitable device. As shown in the plot, the sensitivities (expressed as a percentage of a steady state sensitivity) of each sensor, as measured, are very close to sensitivities of other sensors in the group at any given time of the sensor session. While not wishing to be bound by theory, it is believed that the observed upward trend in sensitivity (over time), which is particularly pronounced in the early part of the sensor session, can be attributed to conditioning and hydration of sensing regions of the working electrode. It is also believed that the glucose concentration of the fluid surrounding the continuous glucose sensor during startup of the sensor can also affect the sensitivity drift.

With the sensors tested in the example study illustrated by FIG. 9, the change in sensor sensitivity (expressed as a percentage of a substantially steady state sensitivity), over a time defined by a sensor session, resembled a logarithmic growth curve. It should be understood that other continuous analyte sensors fabricated with different techniques, with different specifications (e.g., different membrane thickness or composition), or under different manufacturing conditions, may exhibit a different sensor sensitivity profile (e.g., one associated with a linear function). Nonetheless, with improved control over operating conditions of the sensor fabrication process, high levels of reproducibility may be achieved, such that sensitivity profiles exhibited by individual sensors of a sensor population (e.g., a sensor lot) are substantially similar and sometimes nearly identical.

In various examples, the change or drift in sensitivity over a sensor session is not only substantially consistent among sensors manufactured in substantially the same way under substantially same conditions, but also that modeling can be performed through mathematical functions that can accurately estimate this change or drift. As illustrated in FIG. 9, an estimative algorithm function 920 can be used to define the relationship between time during the sensor session and sensor sensitivity. The estimative algorithm function may be generated by testing a sample set (comprising one or more sensors) from a sensor lot under in vivo and/or in vitro conditions. Alternatively, the estimative algorithm function may be generated by testing each sensor under in vivo and/or in vitro conditions.

In some examples, a sensor may undergo an in vitro sensor sensitivity drift test, in which the sensor is exposed to changing conditions (e.g., step changes of glucose concentrations in a solution), and an in vitro sensitivity profile of the sensor is generated over a certain time period. The time period of the test may substantially match an entire sensor session of a corresponding in vivo sensor, or it may encompass a portion of the sensor session (e.g., the first day, the first two days, or the first three days of the sensor session, etc.). It is contemplated that the above-described test may be performed on each individual sensor, or alternatively on one or more sample sensors of a sensor lot. From this test, an in vitro sensitivity profile may be created, from which an in vivo sensitivity profile may be modeled and/or formed.

From the in vivo or in vitro testing, one or more data sets, each comprising data points associating sensitivity with time, may be generated and plotted. A sensitivity profile or curve can then be fitted to the data points. If the curve fit is determined to be satisfactory (e.g., if the standard deviation of the generated data points is less a certain threshold), then the sensor sensitivity profile or curve may be judged to have passed a quality control and suitable for release. From there, the sensor sensitivity profile can be transformed into an estimative algorithm function or alternatively into a look-up table. The algorithm function or look-up table can be stored in a computer-readable memory, for example, and accessed by a computer processor.

The estimative algorithm function may be formed by applying curve fitting techniques that regressively fit a curve to data points by adjusting the function (e.g., by adjusting constants of the function) until an optimal fit to the available data points is obtained. For example, a “curve” (i.e., a function sometimes referred to as a “model”) may be fitted and generated that relates one data value to one or more other data values and selecting parameters of the curve such that the curve estimates the relationship between the data values. By way of example, selection of the parameters of the curve may involve selection of coefficients of a polynomial function. In some examples, the curve fitting process may involve evaluating how closely the curve determined in the curve fitting process estimates the relationship between the data values, to determine the optimal fit. The term “curve,” as used herein, is a broad term, and is to be given its ordinary and customary meaning to a person of ordinary skill in the art (and is not to be limited to a special or customized meaning), and refers to a function or a graph of a function, which can involve a rounded curve or a straight curve, i.e., a line.

The curve may be formed by any of a variety of curve fitting techniques, such as, for example, the linear least squares fitting method, the non-linear least squares fitting method, the Nelder-Mead Simplex method, the Levenberg-Marquardt method, and variations thereof. In addition, the curve may be fitted using any of a variety of functions, including, but not limited to, a linear function (including a constant function), logarithmic function, quadratic function, cubic function, square root function, power function, polynomial function, rational function, exponential function, sinusoidal function, and variations and combinations thereof. For example, in some examples, the estimative algorithm comprises a linear function component which is accorded a first weight w1, a logarithmic function component which is accorded a second weight w2, and an exponential function component which is accorded a third weight w3. In further examples, the weights associated with each component can vary as a function of time and/or other parameters, but in alternative example, one or more of these weights are constant as a function of time.

In various examples, the estimative algorithm function's correlation (e.g., R2 value), which is a measure of the quality of the fit of the curve to the data points, with respect to data obtained from the sample sensors, may be one metric used to determine whether a function is optimal. In various examples, the estimative algorithm function formed from the curve fitting analysis may be adjusted to account for other parameters, e.g., other parameters that may affect sensor sensitivity or provide additional information about sensor sensitivity. For example, the estimative algorithm function may be adjusted to account for the sensitivity of the sensor to hydrogen peroxide or other chemical species.

Estimative algorithms formed and used to accurately estimate an individual sensor's sensitivity, at any time during a sensor session, can be based on factory calibration and/or based on a single early reference measurement (e.g., using a single point blood glucose monitor). In some examples, sensors across a population of continuous analyte sensors manufactured in substantially the same way under substantially same conditions exhibit a substantially fixed in vivo to in vitro sensitivity relationship. For example, in one example, the in vivo sensitivity of a sensor at a certain time after start of sensor use (e.g., at t=about 5, 10, 15, 30, 60, 120, or 180 minutes after sensor use) is consistently equal to a measured in vitro sensitivity of the sensor or of an equivalent sensor. From this relationship, an initial value of in vivo sensitivity can be generated, from which an algorithmic function corresponding to the sensor sensitivity profile can be formed. In some examples, from this initial value (which represents one point in the sensor sensitivity profile), the rest of the entire sensor sensitivity profile can be determined and plotted. The initial value of in vivo sensitivity can be associated with any portion of the sensor sensitivity profile. In various examples, multiple initial values of in vivo sensitivities, which are time-spaced apart, and which correspond to multiple in vitro sensitivities, can be calculated and combined together to generate the sensor sensitivity profile.

With some examples, it has been found that not only does the sensor's sensitivity tend to drift over time, but that the sensor's baseline also drifts over time. Accordingly, in various examples, the concepts behind the methods and systems used to predict sensitivity drift can also be applied to create a model that predicts baseline drift over time. Although not wishing to be bound by theory, it is believed that the total signal received by the sensor electrode is comprised of a glucose signal component, an interference signal component, and an electrode-related baseline signal component that is a function of the electrode and that is substantially independent of the environment (e.g., extracellular matrix) surrounding the electrode. As noted above, the term “baseline,” as used herein, refers without limitation to the component of an analyte sensor signal that is not related to the analyte concentration. Accordingly, the baseline, as the term is defined herein, is comprised of the interference signal component and the electrode-related baseline signal component. Again, while not wishing to be bound by theory, it is believed that increased membrane permeability typically not only results in an increased rate of glucose diffusion across the sensor membrane, but also in an increased rate of diffusion of interferents across the sensor membrane. Accordingly, changes in sensor membrane permeability over time, which causes sensor sensitivity drift, will similarly also likely cause the interference signal component of the baseline to drift. Simply put, the interference signal component of the baseline is not static, and is typically changing as a function of time, which, in turn, causes the baseline to also drift over time. By analyzing how each of the aforementioned components of the baseline reacts to changing conditions and to time (e.g., as a function of time, temperature), a predictive model can be developed to predict how the baseline of a sensor will drift during a sensor session. By being able to prospectively predict both sensitivity and baseline of the sensor, it is believed that a factory calibrated or automatically self-calibrating continuous analyte sensor can be achieved, i.e., a sensor that does not require use of reference measurements (e.g., a finger stick measurement) for calibration.

### Calibration Code

The process of manufacturing continuous analyte sensors may sometimes be subjected to a degree of variability between sensor lots, as will be described in greater detail below. To compensate for this variability, one or more calibration codes may be assigned to each sensor or sensor set to define parameters that can affect sensor sensitivity or provide additional information about the sensitivity profile. The calibration codes can reduce variability in the different sensors, ensuring that the results obtained from using sensors from different sensors lots will be generally equal and consistent by applying an algorithm that adjusts for the differences. In one example, the analyte sensor system may be configured such that one or more calibration codes are to be manually entered into the system by a user. In other examples, the calibration codes may be part of a calibration encoded label that is adhered to (or inserted into) a package of multiple sensors. The calibration encoded label itself may be read or interrogated by any of a variety of techniques, including, but not limited to, optical techniques, RFID (radio-frequency identification), or the like, and combinations thereof. These techniques for transferring the code to the sensor system may be more automatic, accurate, and convenient for the patient, and less prone to error, as compared to manual entry. Manual entry, for instance, possesses the inherent risk of an error caused by a patient or hospital staff entering the wrong code, which can lead to an incorrect calibration, and thus inaccurate glucose concentration readings. In turn, this may result in a patient or hospital staff taking an inappropriate action (e.g., injecting insulin while in a hypoglycemic state).

In some examples, calibration codes assigned to a sensor may include a first calibration code associated with a predetermined logarithmic function corresponding to a sensitivity profile, a second calibration code associated with an initial in vivo sensitivity value, and other calibration codes, with each code defining a parameter that affects sensor sensitivity or provides information about sensor sensitivity. The other calibration codes may be associated with any a priori information or parameter described elsewhere herein and/or any parameter that helps define a mathematical relationship between the measured signal and analyte concentration. The calibration code may be developed from these measurements or may be developed based on manufacturing parameters known, determined, or measured during fabrication of, e.g., a lot, or by a combination of these.

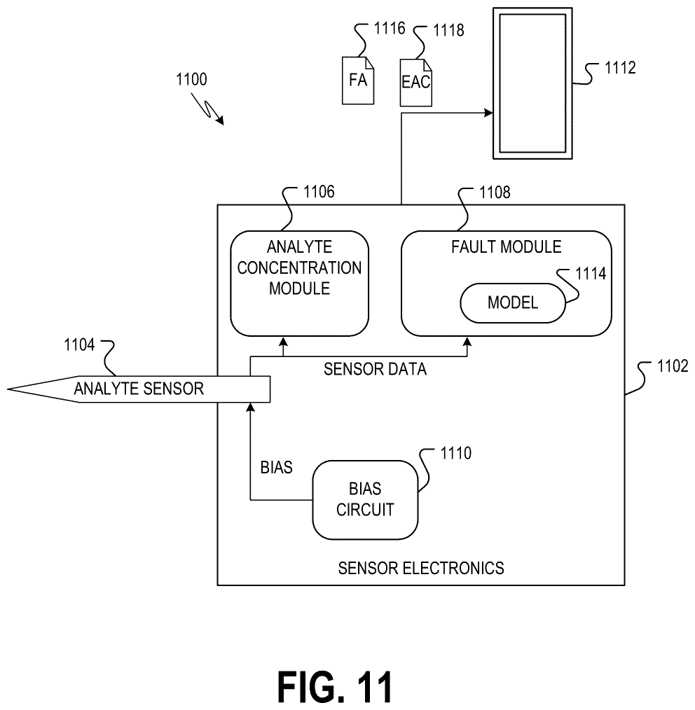

FIG. 11 is a diagram showing one example of an analyte sensor system 1100. The analyte sensor system 1100, in various examples, may be arranged in a manner similar to that of the analyte sensor system 102 and/or the analyte sensor system 800 described herein. The analyte sensor system 1100 may be configured to detect and respond to a potential sensor fault. For example, the analyte sensor system 1100 may be configured to detect and respond when an analyte sensor 1104 enters and end-of-life state or other fault condition.

The analyte sensor system 1100 comprises the analyte sensor 1104 and sensor electronics 1102. The analyte sensor 1104 may be arranged, for example, as described herein. The sensor electronics 1102 may include a bias circuit 1110, an analyte concentration module 1106, and a fault module 1108. The bias circuit 1110 may comprise various power supplies, regulators, and/or the like to generate a bias signal that is provided to bias the analyte sensor 1104, for example, as described herein.

Sensor data generated by the analyte sensor 1104 may be provided to the analyte concentration module 1106 and the fault module 1108. The analyte concentration module 1106 and fault module 1108 may be or include an executable code that may be executed by a processor of the sensor electronics 1102. For example, the analyte concentration module 1106 may be executed to receive the sensor data and generate corresponding estimated analyte concentration data 1118. The estimated analyte concentration data 1118 may be provided to a display 1112, which may display the estimated analyte concentration to a host or other user. The display 1112 may be any suitable device having a wired and/or wireless connection to the sensor electronics 1102. In some examples, the display 1112 is or includes one or more of the devices 112, 114, 116, 118, 122, 128, 132 described herein.

The fault module 1108 may be executed to access the sensor data generated by the analyte sensor 1104 and detect when the analyte sensor 1104 has entered an end-of-life state or other fault condition. In some examples, the fault module 1108 executes a trained computerized model 1114. The trained computerized model 1114 may receive some or all of the sensor data as input and may generate an output indicative of whether the analyte sensor is currently experiencing a fault. The fault may be a temporary fault and/or may be associated with and end-of-life state.

If the trained computerized model 1114 generates an output indicative of an end-of-life state or other fault condition at the analyte sensor 1104, the sensor electronics 1102 may take a responsive action. In some examples, the responsive action includes providing a fault alert 1116 to the display 1112. The display 1112 may provide an output indicative of the fault alert 1116 to alert a host or other user of the existence of the end-of-life state or other fault condition. In some examples, the responsive action includes disabling the display 1112 and/or preventing the display from indicating the estimated analyte concentration data 1118. This may prevent the host or other user from relying on estimated analyte concentration data 1118 generated by an analyte sensor 1104 that is in an end-of-life state or other fault condition. Also, in some examples, the responsive action may include ending a sensor session for the analyte sensor 1104. This may include, for example, ceasing to provide the bias signal to the analyte sensor 1104.

The trained computerized model 1114 may be any suitable trained computerized model, such as a neural network. A neural network is a type of computerized model that is constructed from interconnected cells divided into layers. Each cell is implemented as a function executed by the sensor electronics or other processor or computing system. The sensor electronics or other system implements a cell by processing cell input data and generating corresponding cell output data according to the cell's function. In some examples, the cells of the neural network are arranged in layers. An input layer of cells receives an input to the neural network. The inputs to cells making up the intermediate layers may include outputs of cells in the input layer and/or outputs of other cells in the intermediate layers. Cells in an output layer receive inputs generated, for example, by cells in the intermediate layers and generate outputs. The outputs of the cells of the output layer may represent the output of the neural network.

A neural network may be trained utilizing labeled training data to generate an indication of whether the analyte sensor 1104 is in an end-of-life state or other fault condition. Labeled training data comprises instances of input data and a corresponding output. In the context of a continuous analyte sensor, for example, labeled training data may comprise sensor signal data describing the sensor signal over a time period and a corresponding label indicating a state of the analyte sensor, such as whether the sensor signal data describes an analyte sensor that is in and end-of-life state or otherwise experiencing a fault.

Training may be conducted in a series of training epochs. In each training epoch, some or all of the training data may be provided as input to the neural network. The output of the neural network may be compared to the corresponding label or labels to determine a loss of the neural network. The loss describes a difference between the output of the neural network and the label or labels indicating the correct output for the corresponding input. The loss is used to modify the neural network, for example, by modifying weights or other portions of the functions implemented by the individual cells. In some examples, a predetermined number of training epochs are performed. Also, in some examples, training epochs are performed until the loss of the neural network meets a threshold. Various different techniques may be used to determine the loss and to determine updates to neural network cells during a training epoch. Example techniques include, gradient descent and back propagation.

In some examples, the trained computerized model 1114 is or includes a Recurrent Neural Network (RNN). An RNN is a bidirectional neural network. For example, some cells of the RNN may receive input that is based on the outputs of previous cells and may, additionally or alternatively, receive input that depends on a previous output of the same cell. An example RNN is an LSTM or LSTM layer. Also, in some examples, the trained computerized model 1114 is or includes a Convolutional Neural Network (CNN). A CNN is a unidirectional or feed forward neural network in which the inputs to the cells are based on the outputs of cells from prior layers.

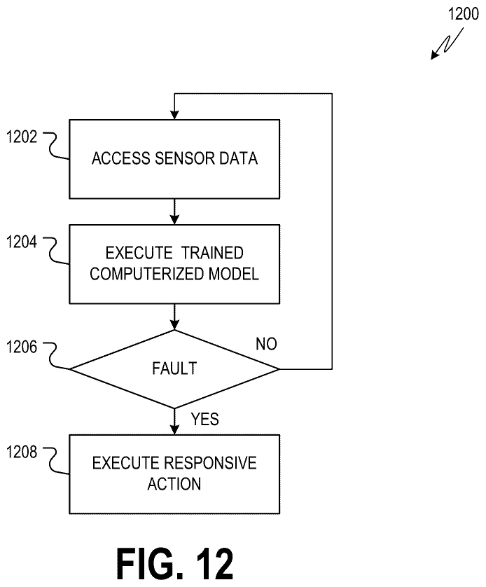

FIG. 12 is a flowchart showing one example of a process flow 1200 that may be executed in an analyte sensor system, such as the analyte sensor system 1100 of FIG. 11, to detect and respond to potential sensor fault. For example, the process flow 1200 may be executed to detect and respond when an analyte sensor enters an end-of-life state or other fault condition.

At operation 1202, the analyte sensor system may access sensor data generated by the analyte sensor. In some examples, the sensor data may be accessed continuously as it is generated by the analyte sensor. The analyte sensor system may store some or all of the accessed sensor data as it is generated by the analyte sensor. As a result, the analyte sensor system may store sensor data over a time period. In some examples, the time period begins at the outset of a sensor session for the analyte sensor and may extend to the time at which the analyte sensor output was most recently sampled. Also, in some examples, the time period extends for a predetermined time back from when the analyte sensor was most recently sampled (e.g., a previous 48 hours, a previous 36 hours, a previous 24 hours, a previous 12 hours, a previous 6 hours, a previous hour, and/or the like).

The analyte sensor system, at operation 1204, may use some or all of the accessed sensor data to execute the trained computerized model, for example, as described herein. An output of the trained computerized model may be indicative of whether the analyte sensor is in an end-of-life state or other fault condition. In some examples, the output of the trained computerized model may also be indicative of a type of fault condition of the analyte sensor 1104. For example, the output of the trained computerized model may discriminate between whether the analyte sensor 1104 is in an end-of-life state or whether it is in a recoverable fault condition.

At operation 1206, the sensor electronics determines whether the output of the trained computerized model indicated that the analyte sensor was in an end-of-life state or other fault condition. If the analyte sensor is not in an end-of-life state or other fault condition, the sensor electronics may return to operation 1202 and access additional sensor data.

If the sensor electronics determines at operation 1206 that the analyte sensor is in an end-of-life state or other fault condition, the sensor electronics may execute a responsive action at operation 1208. The responsive action may include, for example, providing a fault alert to a display associated with the analyte sensor system, ceasing the display of analyte concentration data derived from the analyte sensor, ending a sensor session, ceasing to provide a biased signal to the analyte sensor, and/or the like.

In some examples, the responsive action executed at operation 1208 may depend on the type of fault condition indicated by the output of the trained computerized model. For example, if the output of the trained computerized model indicates that the analyte sensor is in an end-of-life state, the sensor electronics may terminate the current sensor session. This may involve, for example, ceasing to provide a biased signal to the analyte sensor. It may also involve providing to the display a fault alert indicating that the sensor session has been terminated. If the output of the trained computerized model indicates that the analyte sensor is in a fault condition that is recoverable, then the sensor electronics may not end the sensor session. For example, the sensor electronics may send a fault alert indicating a temporary fault and/or may suspend display of estimated analyte concentrations derived from the analyte sensor for a predetermined time and/or until the output of the trained computerized model no longer indicates a fault condition.

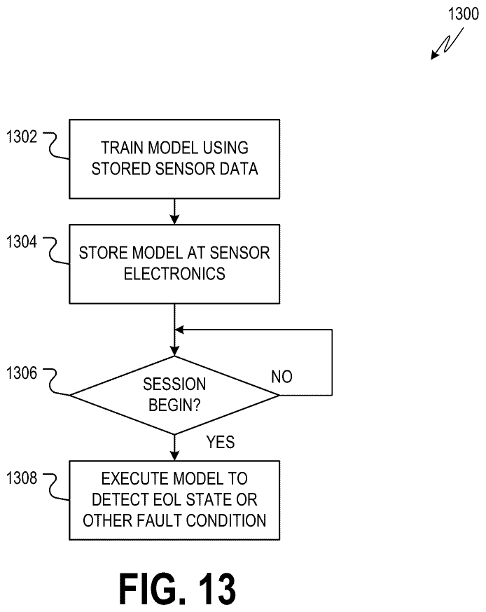

FIG. 13 is a flowchart showing one example of a process flow 1300 that may be executed to deploy an analyte sensor system. In some examples, the process flow 1300 may be executed before and/or during a manufacturing process for the analyte sensor system.

At operation 1302, a computerized model may be trained to detect when an analyte sensor is in an end-of-life state or other error condition. In some examples, the operation 1302 may be performed by a server system, such as the server system 126. The computerized model may be trained in any suitable manner, for example, as described herein. At operation 1304, the trained computerized model is stored at the sensor electronics of an analyte sensor system.

At operation 1306, the sensor electronics may determine if a sensor session has begun for the analyte sensor system. For example, the sensor electronics may detect sensor insertion, as described herein. Sensor insertion may indicate the beginning of a sensor session. If the sensor session has not begun, the sensor electronics may return to operation 1306 and continue to wait for the sensor session to begin. If the sensors session has begun, then the sensor electronics may execute the trained computerized model at operation 1308 to detect when the analyte sensor is in an end-of-life state or other fault condition. For example, the process flow 1200 shows one example way of executing the operation 1308.

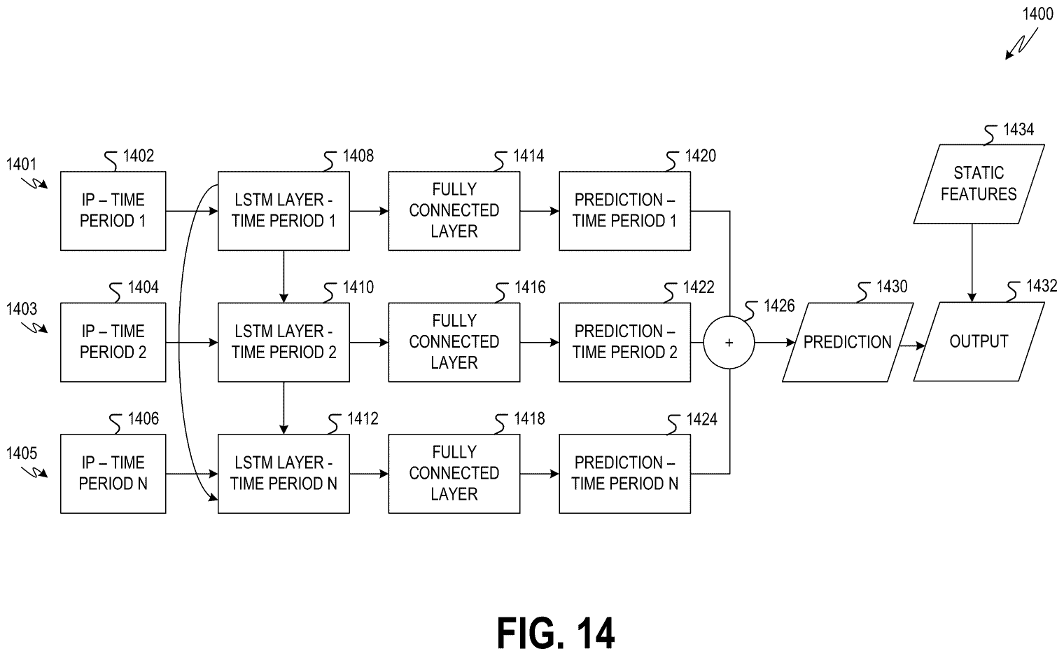

FIG. 14 is a diagram showing an example architecture 1400 of a trained computerized model for determining whether an analyte sensor is in an end-of-life state or other error condition. The architecture 1400 shows an RNN model comprising various LSTM layers 1408, 1410, 1412. For example, the architecture 1400 shows a hierarchal structure comprising three levels 1401, 1403, and 1405. A first level 1401 may comprise various layers 1402, 1408, 1414, 1420 that operate on input data gathered from the analyte sensor over a first time period. A second level 1403 may comprise various layers 1404, 1410, 1416, 1422 that operate on input data gathered from the analyte sensor over a second time period. A third level 1405 may comprise various layers 1406, 1412, 1418, 1424 that operate on input data gathered from the analyte sensor over a third time period, with the first time period, second time period, and third time period being distinct from one another.

In some examples, the first, second, and third time periods may start at different times. For example, the start time of the first time period may be before the start time of the second time period. The start time of the second time period may be before the start time of the third time period, and so on. In some examples, the first time period, the second time period, and the third time may all extend to a most recent sample of the analyte sensor. That is, sensor data generated by the analyte sensor during the first time period, during the second time period, and during the third time period may each include a most recent sample of the analyte sensor. In other examples, one or more of the time periods may exclude one or more most recent samples from the analyte sensor. In some examples, the third time period may be shorter than the second time period and the second time period may be shorter than the first time period.

Consider an example in which the first time period is 12 hours, the time period for the second level 1403 is 20 minutes, and the time period for the third level 1405 may be 30 seconds. In this example, the 12 hours of the first time period may be the most recently elapsed 12 hours, the 20 minutes of the second time period may be the most recently elapsed 20 minutes, and the 30 seconds of the third time period may be the most recently elapsed 30 seconds. In other examples, the time period for the first level 1401 and/or for the second level 1403 may not extend to the present (e.g., may not include the most recently sampled value or values from the analyte sensor).\

The levels 1401, 1403, 1405 include various layers. Each layer of a level 1401, 1403, 1405 may include a number of cells. Cells in a layer may receive inputs from model inputs (e.g., input layers 1402, 1404, 1406), and/or from the outputs of other cells. In some examples, cells in a given layer may receive inputs based on the outputs of cells in a preceding layer. In some examples, as described herein, cells in a given layer may receive inputs that are based on the outputs of preceding or succeeding cells, for example, as described herein such as in a unidirectional or bidirectional RNN.

The input layers 1402, 1404, 1406 of the respective levels 1401, 1403, 1405 may receive as input sensor data generated by the analyte sensor over the time periods of the respective levels 1401, 1403, 1405. The cells of the input layers 1402, 1404, 1406 may generate outputs that are provided as inputs to the respective LSTM layers 1408, 1410, 1412. In some examples, the respective input layers 1402, 1404, 1406 may be fully connected to the corresponding LSTM layers 1408, 1410, 1412. For example, the output of each cell of the input layer 1402 may be provided as input to each cell of the LSTM layer 1408, the output of each cell of the input layer 1404 may be provided as input to each cell of the LSTM layer 1410, and the output of each cell of the input layer 1406 may be provided as input to each cell of the LSTM layer 1412.

The LSTM layers 1408, 1410, 1412 may receive as input the outputs of their respective input layers 1402, 1404, 1406 as well as outputs of the other respective LSTM layers 1408, 1410, 1412. The LSTM layers 1408, 1410, 1412 may comprise cells that are unidirectional, or bidirectional. For example, the inputs to some or all of the cells of the respective LSTM layers 1408, 1410, 1412 may include outputs of one or more subsequent and/or preceding layers. Cells in the respective LSTM layers 1408, 1410, 1412 may generate a cell state, and a hidden state. The cell state is the output of the cell that may be provided to cells of other layers, for example, as described herein. The hidden state is an indicator of information about past data, in this example, past samples of the analyte sensor. The cell state may be determined based on the cells hidden state. As shown in FIG. 14, the LSTM layers 1412 may receive as input the outputs of cells in the LSTM layers 1408, 1410, but not vice versa. The LSTM layers 1410 may receive as input the outputs of cells in the LSTM layers 1408, but not vice versa. The output provided to cells of the other LSTM layers 1408, 1410, 1412 may include the cell output, the hidden output, and/or both.

Although the respective levels 1401, 1403, 1405 comprise single respective LSTM layers 1402, 1404, 1406 in the example of FIG. 14, it will be appreciated that different implementations may include different numbers of LSTM layers. In the example of FIG. 14, each level 1401, 1403, 1405 also comprises a respective fully connected layer 1414, 1416, 1418. Fully connected layers 1414, 1416, 1418 may be multilayer perceptron or convolutional layers comprising cells such that each cell receives the output of each cell of the corresponding LSTM layer 1408, 1410, 1412. Although each level 1401, 1403, 1405 comprises a single respective fully connected layer 1414, 1416, 1418 in FIG. 14, it will be appreciated that some examples may include multiple fully connected layers.

In the example of FIG. 14, the outputs of cells in the respective fully connected layers 1414, 1416, 1418 are provided to respective output prediction layers 1420, 1422, 1424. In some examples, the output prediction layers 1420, 1422, 1424 generate outputs representing a predicted state of the analyte sensor based on the sensor data from the time periods corresponding to the respective levels 1401, 1403, 1405. For example, the output prediction layer 1420 may generate a predicted state of the analyte sensor based on the sensor data from the first time period; the output prediction layer 1422 may generate a predicted state of the analyte sensor based on the sensor data from the second time period; and the output prediction layer 1424 may generate a predicted state of the analyte sensor based on the sensor data from the third time period.

The predicted states of the analyte sensor determined by the output prediction layers 1420, 1422, 1424 may be consolidated at a consolidation block 1426 to generate prediction 1430. The sensor electronics or other suitable processor may execute the consolidation block 1426 by combining the predicted states of the analyte sensor. Any suitable combination may be used such as, for example, summing, averaging, and/or the like. The prediction 1430 may be a predicted state of the analyte sensor based on the outputs of the output prediction layers 1420, 1422, 1424. For example, the prediction 1430 may indicate whether the analyte sensor is in an end-of-life state or other fault condition. The prediction 1430 may be used to generate an output 1432 based on static features 1434. Static features are info that does not change with time. For example, sensor manufacturing features, patient demographics, etc.

This hierarchical architecture works more sufficiently for very long sequence of time series. The sensor signal we collected usually consists of tens of thousands of samples for a single sensor, and this hierarchical architecture deals with signal from different time window separately and then combine them together. As a result, it can capture signal behavior of low granularity and high granularity at the same time. In comparison to regular RNN or LSTM, this hierarchical network has less tendency to suffer gradient explosion/vanishing problem which will block information of long time ago from passing through the recurrent chain.

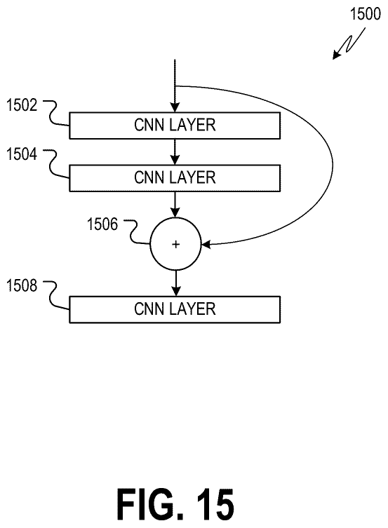

In some examples, the computerized model may include one or more CNN layers comprising one or more a residual blocks. In a residual block, cells in a CNN layer receive as input the output of cells from a previous CNN layer as well as an input provided to one or more previous CNN layers. FIG. 15 is a diagram 1500 showing a portion of a computerized model including CNN layers 1502, 1504, 1508. The CNN layer 1502 receives an input that may be, for example, an input to the model and/or an output of a preceding layer. The output of the CNN layer 1502 is provided to the CNN layer 1504. For example, the outputs of cells in the CNN layer 1502 are provided as input to cells of the CNN layer 1504.

The outputs of the respective cells of the CNN layer 1504 may be combined with the input to the CNN layer 1502 at combination block 1506 to generate an input to the CNN layer 1508. Any suitable combination may be performed at combination block 1506 including, for example, an average, a sum, and/or the like. The output of the CNN layer 1508 may be provided as input to a subsequent layer and/or taken as output of the model. In various examples, the residual block arrangement shown in FIG. 15 may be used in a CNN computerized model and/or may be incorporated into computerized models utilizing CNN and elements.

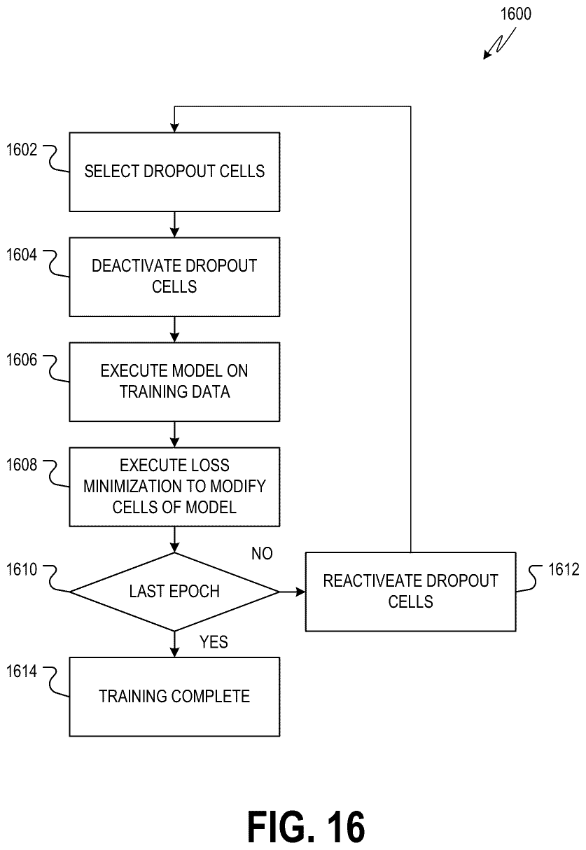

FIG. 16 is a flowchart showing one example of a process flow 1600 that may be executed to train a computerized model utilizing dropout. The process flow 1600 may be executed, for example, by a server system, such as the server system 126, to train a computerized model to predict whether an analyte sensor is in an end-of-life state or other error condition. The process flow 1600 may be executed to train a computerized model comprising one or more neural network layers, as described herein.

At operation 1602, the server system may select cells from the computerized model for dropout. In some examples, the dropout cells may be selected randomly or pseudo-randomly from among the cells of the computerized model. The number of dropout cells selected may be predetermined. In some examples, the number of dropout cells selected may be a parameter that may be set by a user of the server system.

At operation 1604, the server system may deactivate the dropout cells. Deactivating the dropout cells may comprise modifying the dropout cells to act as pass-throughs. For example, inputs to the dropout cells may be passed as input to the sale or cells that would receive outputs of the dropout cells. In some examples, deactivating the dropout cells may include modifying code that is executed to execute the training model.

At operation 1606, the server system may execute the computerized model on labeled training data with the dropout cells deactivated. For example, the server system may execute the computerized model using training data as input. The label associated with the training data may describe the desired output corresponding to the training data. For example, the training data may include sensor data generated by one or more analyte sensors. The label may indicate a state of the analyte sensor that generated the data (e.g., whether the analyte sensor was in an end-of-life state or other fault condition). At operation 1608, the server system may execute a loss minimization technique to modify the cells of the computerized model. This may be performed according to any suitable technique, such as, for example, gradient descent and backpropagation.

At operation 1610, the server system may determine if the current training epoch is the final training epoch. As described herein, the number of training epochs performed may be determined in various different ways. For example, the server system may be programmed to perform a predetermined number of training epochs. Also, in some examples, the server system may perform training epochs until the loss, for example, determined at operation 1608, meets a threshold. If the current training epoch is the final training epoch, the training of the model may be complete at operation 1614. If the current training epoch is not the last training epoch, then the server system may reactivate the dropout cells at operation 1612 and return to operation 1602 to select new dropout cells for a next training epoch.

In some examples, training a computerized model using dropout, as described in FIG. 16, may generate a trained computerized model that is less likely to over fit to the training data. Also, it will be appreciated that FIG. 16 shows an arrangement in which dropout cells are selected and deactivated in every training epoch. In some examples, dropout may be performed for some training epochs, but not others.

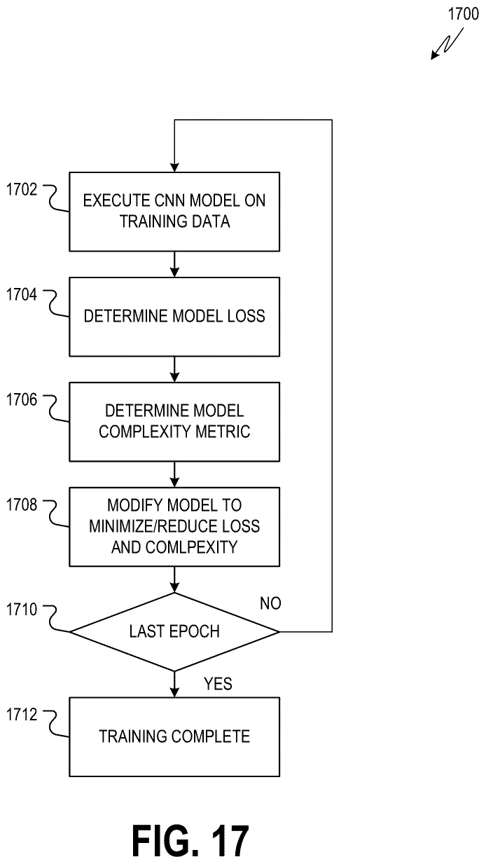

FIG. 17 is a flowchart showing one example of a process flow 1700 that may be executed to train a computerized model considering model loss as well as complexity. For example, the process flow 1700 may implement an L2 regularization which adds sum of squares of some of parameters in the model to the loss function. The process flow 1700 may be executed, for example, by a server system, such as the server system 126, to train a computerized model to predict whether an analyte sensor is in an end-of-life state or other error condition.

At operation 1702, the server system may execute the computerized model on labeled training data. At operation 1704, the server system may determine a loss of the model based on the results of executing the model at operation 1702. For example, the server system may compare an output of the model to one or more labels associated with the training data. The loss of the model may indicate a difference between the output generated by the model and the output that should be generated by the model in view of the training data label.

At operation 1706, the server system may determine a model complexity metric. The model complexity metric may describe a complexity of the model. In some examples, the complexity metric may be based on the values of the weights applied by the various cells of the computerized model. Also, in some examples, the complexity metric may be based on the number of cells in the model having a non-zero weight.

At operation 1708, the server system may modify the model to minimize and/or reduce both the loss and the complexity metric. For example, the server system may implement a gradient descent and back prop in technique with respect to both loss and complexity. At operation 1710, the server system may determine if the current training epoch is the last training epoch. If the current training epoch is the last training epoch, then the training of the computerized model may be complete at operation 1712. If the current training epoch is not the last training epoch, then the server system may return to operation 1702 to execute an additional training epoch.

In some examples, training a neural network using L2 regularization, for example, as described by the process flow 1700, may reduce the risk of over fitting the training data. Also, it will be appreciated that the various model training examples described herein may be combined. For example, the server system may utilize both dropout and L2 regularization when training a computerized model.

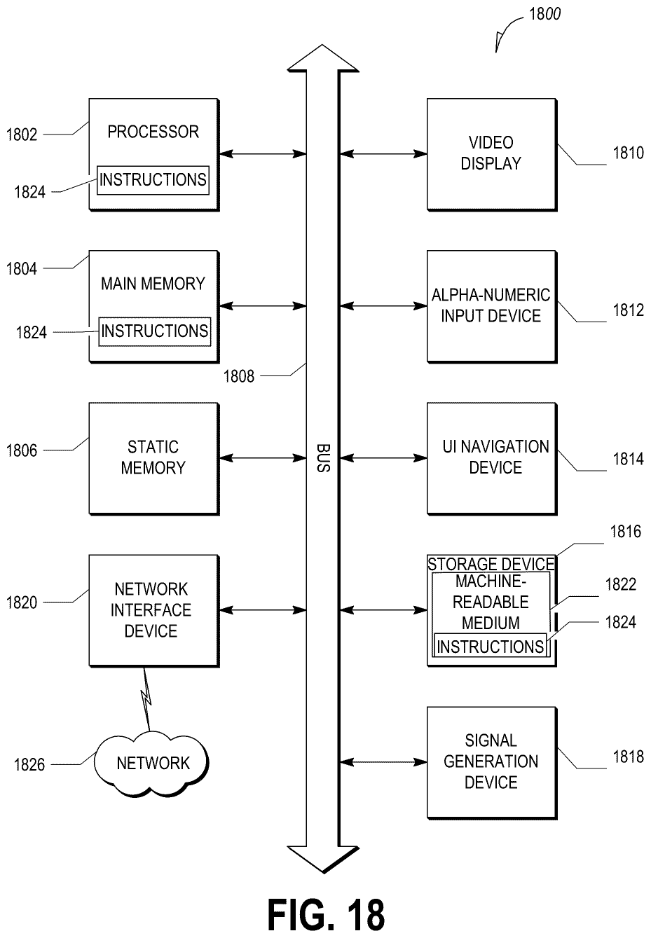

FIG. 18 is a block diagram illustrating a computing device hardware architecture 1800, within which a set or sequence of instructions can be executed to cause a machine to perform examples of any one of the methodologies discussed herein. The hardware architecture 1800 can describe various computing devices including, for example, the sensor electronics 106, the peripheral medical device 122, the smart device 112, the tablet 114, etc.

The architecture 1800 may operate as a standalone device or may be connected (e.g., networked) to other machines. In a networked deployment, the architecture 1800 may operate in the capacity of either a server or a client machine in server-client network environments, or it may act as a peer machine in peer-to-peer (or distributed) network environments. The architecture 1800 can be implemented in a personal computer (PC), a tablet PC, a hybrid tablet, a set-top box (STB), a personal digital assistant (PDA), a mobile telephone, a web appliance, a network router, a network switch, a network bridge, or any machine capable of executing instructions (sequential or otherwise) that specify operations to be taken by that machine.

The example architecture 1800 includes a processor unit 1802 comprising at least one processor (e.g., a central processing unit (CPU), a graphics processing unit (GPU), or both, processor cores, compute nodes). The architecture 1800 may further comprise a main memory 1804 and a static memory 1806, which communicate with each other via a link 1808 (e.g., bus). The architecture 1800 can further include a video display unit 1810, an input device 1812 (e.g., a keyboard), and a UI navigation device 1814 (e.g., a mouse). In some examples, the video display unit 1810, input device 1812, and UI navigation device 1814 are incorporated into a touchscreen display. The architecture 1800 may additionally include a storage device 1816 (e.g., a drive unit), a signal generation device 1818 (e.g., a speaker), a network interface device 1820, and one or more sensors (not shown), such as a Global Positioning System (GPS) sensor, compass, accelerometer, or another sensor.

In some examples, the processor unit 1802 or another suitable hardware component may support a hardware interrupt. In response to a hardware interrupt, the processor unit 1802 may pause its processing and execute an Interrupt Service Routine (ISR), for example, as described herein.

The storage device 1816 includes a machine-readable medium 1822 on which is stored one or more sets of data structures and instructions 1824 (e.g., software) embodying or used by any one or more of the methodologies or functions described herein. The instructions 1824 can also reside, completely or at least partially, within the main memory 1804, within the static memory 1806, and/or within the processor unit 1802 during execution thereof by the architecture 1800, with the main memory 1804, the static memory 1806, and the processor unit 1802 also constituting machine-readable media.

### Executable Instructions and Machine-Storage Medium

The various memories (i.e., 1804, 1806, and/or memory of the processor unit(s) 1802) and/or storage device 1816 may store one or more sets of instructions and data structures (e.g., instructions) 1824 embodying or used by any one or more of the methodologies or functions described herein. These instructions, when executed by processor unit(s) 1802 cause various operations to implement the disclosed examples.

As used herein, the terms “machine-storage medium,” “device-storage medium,” “computer-storage medium” (referred to collectively as “machine-storage medium 1822”) mean the same thing and may be used interchangeably in this disclosure. The terms refer to a single or multiple storage devices and/or media (e.g., a centralized or distributed database, and/or associated caches and servers) that store executable instructions and/or data, as well as cloud-based storage systems or storage networks that include multiple storage apparatus or devices. The terms shall accordingly be taken to include, but not be limited to, solid-state memories, and optical and magnetic media, including memory internal or external to processors. Specific examples of machine-storage media, computer-storage media, and/or device-storage media 1822 include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms machine-storage media, computer-storage media, and device-storage media 1822 specifically exclude carrier waves, modulated data signals, and other such media, at least some of which are covered under the term “signal medium” discussed below.

### Signal Medium

The term “signal medium” or “transmission medium” shall be taken to include any form of modulated data signal, carrier wave, and so forth. The term “modulated data signal” means a signal that has one or more of its characteristics set or changed in such a matter as to encode information in the signal.

### Computer-Readable Medium

The terms “machine-readable medium,” “computer-readable medium” and “device-readable medium” mean the same thing and may be used interchangeably in this disclosure. The terms are defined to include both machine-storage media and signal media. Thus, the terms include both storage devices/media and carrier waves/modulated data signals.

The instructions 1824 can further be transmitted or received over a communications network 1826 using a transmission medium via the network interface device 1820 using any one of a number of well-known transfer protocols (e.g., HTTP). Examples of communication networks include a LAN, a WAN, the Internet, mobile telephone networks, plain old telephone service (POTS) networks, and wireless data networks (e.g., Wi-Fi, 3G, 4G LTE/LTE-A, 5G or WiMAX networks). The term “transmission medium” shall be taken to include any intangible medium that is capable of storing, encoding, or carrying instructions for execution by the machine, and includes digital or analog communications signals or other intangible media to facilitate communication of such software.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Various components are described in the present disclosure as being configured in a particular way. A component may be configured in any suitable manner. For example, a component that is or that includes a computing device may be configured with suitable software instructions that program the computing device. A component may also be configured by virtue of its hardware arrangement or in any other suitable manner.

The above description is intended to be illustrative, and not restrictive. For example, the above-described examples (or one or more aspects thereof) can be used in combination with others. Other examples can be used, such as by one of ordinary skill in the art upon reviewing the above description. The Abstract is to allow the reader to quickly ascertain the nature of the technical disclosure, for example, to comply with 37 C.F.R. § 1.72(b) in the United States of America. It is submitted with the understanding that it will not be used to interpret or limit the scope or meaning of the claims.

Also, in the above Detailed Description, various features can be grouped together to streamline the disclosure. However, the claims cannot set forth every feature disclosed herein, as examples can feature a subset of said features. Further, examples can include fewer features than those disclosed in a particular example. Thus, the following claims are hereby incorporated into the Detailed Description, with each claim standing on its own as a separate example. The scope of the examples disclosed herein is to be determined with reference to the appended claims, along with the full scope of equivalents to which such claims are entitled.

Each of these non-limiting examples in any portion of the above description may stand on its own or may be combined in various permutations or combinations with one or more of the other examples.

The above detailed description includes references to the accompanying drawings, which form a part of the detailed description. The drawings show, by way of illustration, specific embodiments in which the subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” “third,” etc., are used merely as labels, and are not intended to impose numerical requirements on their objects.

Geometric terms, such as “parallel”, “perpendicular”, “round”, or “square” are not intended to require absolute mathematical precision, unless the context indicates otherwise. Instead, such geometric terms allow for variations due to manufacturing or equivalent functions. For example, if an element is described as “round” or “generally round”, a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

What is claimed is:

1. A continuous analyte sensor system comprising:
a continuous analyte sensor; and
sensor electronics in communication with the continuous analyte sensor, the sensor electronics being programmed to perform operations comprising:
accessing sensor data generated by the continuous analyte sensor over a time period;
executing a computerized model based on the sensor data, the computerized model comprising: a first Long Short-Term Memory (LSTM) layer and a second LSTM layer, the executing of the computerized model comprising:
providing a first portion of the sensor data to the first LSTM layer, the first portion of the sensor data being from a first portion of the time period;
providing a second portion of the sensor data to the second LSTM layer, the second portion of the sensor data being from a second portion of the time period, the second portion of the time period being shorter than the first portion of the time period, and the first portion of the time period beginning before the second portion of the time period; and
generating a predicted sensor state based on an output of the first LSTM layer and an output of the second LSTM layer; and
responsive to the predicted sensor state indicating an error condition, modifying a user interface output to suspend display of an output of the continuous analyte sensor.

2. The continuous analyte sensor system of claim 1, the first portion of the time period being about 12 hours and the second portion of the time period being less than about 12 hours.

3. The continuous analyte sensor system of claim 1, the first portion of the time period being about 20 minutes and the second portion of the time period being less than about 20 minutes.

4. The continuous analyte sensor system of claim 1, the first portion of the time period being about 30 seconds.

5. The continuous analyte sensor system of claim 1, the output of the second LSTM layer being based on at least a portion of output of the first LSTM layer.

6. The continuous analyte sensor system of claim 5, the computerized model further comprising a third LSTM layer, the executing of the computerized model further comprising providing a third portion of the sensor data to the third LSTM layer, the third portion of the sensor data being from a third portion of the time period, the third portion of the time period being shorter than the second portion of the time period, and the second portion of the time period beginning more recently than the third portion of the time period.

7. The continuous analyte sensor system of claim 6, the predicted sensor state also being based on an output of the third LSTM layer.

8. The continuous analyte sensor system of claim 7, the output of the third LSTM layer being based on at least a portion of the output of the first LSTM layer and at least a portion of the output of the second LSTM layer.

9. The continuous analyte sensor system of claim 7, the executing of the computerized model further comprising executing a first fully connected layer, the first fully connected layer generating a first fully connected layer output based on the output of the first LSTM layer, the predicted sensor state also being based on the output of the first fully connected layer.

10. The continuous analyte sensor system of claim 1, the operations further comprising, responsive to the predicted sensor state indicating an error condition, terminating a sensor session of the continuous analyte sensor.

11. The continuous analyte sensor system of claim 1, the operations further comprising, responsive to the predicted sensor state indicating an error condition, ceasing to provide a bias to the continuous analyte sensor.

12. A method of operating a continuous analyte sensor, the method comprising:
accessing sensor data generated by the continuous analyte sensor over a time period;
executing a computerized model based on the sensor data, the computerized model comprising: a first Long Short-Term Memory (LSTM) layer and a second LSTM layer, the executing of the computerized model comprising:
providing a first portion of the sensor data to the first LSTM layer, the first portion of the sensor data being from a first portion of the time period;
providing a second portion of the sensor data to the second LSTM layer, the second portion of the sensor data being from a second portion of the time period, the second portion of the time period being shorter than the first portion of the time period, and the first portion of the time period beginning before the second portion of the time period;
generating a predicted sensor state based on an output of the first LSTM layer and an output of the second LSTM layer; and
responsive to the predicted sensor state indicating an error condition, modifying a user interface output to suspend display of an output of the continuous analyte sensor.

13. The method of claim 12, the output of the second LSTM layer being based on at least a portion of output of the first LSTM layer.

14. The method of claim 13, the computerized model further comprising a third LSTM layer, the executing of the computerized model further comprising providing a third portion of the sensor data to the third LSTM layer, the third portion of the sensor data being from a third portion of the time period, the third portion of the time period being shorter than the second portion of the time period, and the second portion of the time period beginning more recently than the third portion of the time period.

15. The method of claim 14, the predicted sensor state also being based on an output of the third LSTM layer.

16. The method of claim 15, the output of the third LSTM layer being based on at least a portion of the output of the first LSTM layer and at least a portion of the output of the second LSTM layer.

17. The method of claim 15, the executing of the computerized model further comprising executing a first fully connected layer, the first fully connected layer generating a first fully connected layer output based on the output of the first LSTM layer, the predicted sensor state also being based on the output of the first fully connected layer.

18. The method of claim 12, further comprising, responsive to the predicted sensor state indicating an error condition, terminating a sensor session of the continuous analyte sensor.

19. The method of claim 12, further comprising, responsive to the predicted sensor state indicating an error condition, ceasing to provide a bias to the continuous analyte sensor.

20. A non-transitory machine-readable medium comprising instructions thereon that, when executed by at least one processor, cause the at least one processor to perform operations comprising:
accessing sensor data generated by a continuous analyte sensor over a time period;
executing a computerized model based on the sensor data, the computerized model comprising: a first Long Short-Term Memory (LSTM) layer and a second LSTM layer, the executing of the computerized model comprising:
providing a first portion of the sensor data to the first LSTM layer, the first portion of the sensor data being from a first portion of the time period;
providing a second portion of the sensor data to the second LSTM layer, the second portion of the sensor data being from a second portion of the time period, the second portion of the time period being shorter than the first portion of the time period, and the first portion of the time period beginning before the second portion of the time period; and
generating a predicted sensor state based on an output of the first LSTM layer and an output of the second LSTM layer; and
responsive to the predicted sensor state indicating an error condition, modifying a user interface output to suspend display of an output of the continuous analyte sensor.
